(19) United States
(12) Patent Application Publication
(10) Pub. No.: US 2004/0248901 A1
(43) Pub. Date: Dec. 9, 2004

(54) Compositions containing itraconazole and their preparation methods

(76) Inventors: Beom Jin Lee, Kangwon-do (KR); Dong Won Lee, Seoul (KR); Choon Young Choi, Kangwon-do (KR)

Correspondence Address: NATH & ASSOCIATES, 1030 15th STREET, NW, 6TH FLOOR, WASHINGTON, DC, 20005, US

(21) Appl. No.: 10/487,014

(22) PCT Filed: Aug. 23, 2002
(86) PCT No.: PCT/KR02/01593

(30) Foreign Application Priority Data
Aug. 27, 2001 (KR) 2001/516630

(51) Int. Cl. A61K 31/496; A61K 9/00

(52) U.S. Cl. 514/254.07; 424/400

(57) ABSTRACT

The present invention relates to compositions containing itraconazole with a greatly increased bioavailability and their preparation methods. More specifically, the present invention relates to compositions containing itraconazole which is a sparingly-soluble drug, fatty acid or fatty alcohol and surfactant and their preparation methods. Compositions according to the present invention act as Self-MicroEmulsifying Drug Delivery System(SMEDDS) wherein itraconazole which is a sparingly-soluble drug is dissolved and dispersed to form a mocoidal phase, and the mocoidal phase is dissolved in water to form microemulsion. And because of increased dissolution property and increased bioavailability, compositions according to the present invention show equal efficacy using less amount than commercial pharmaceutical preparations such as sporanox capsule and are cheaper rather than the commercial pharmaceutical preparations such as sporanox capsule.

### TECHNICAL FIELD

The present invention relates to compositions containing itraconazole with remarkably improved bioavailability, and more particularly, pharmaceutical compositions comprising poorly water-soluble itraconazole, fatty acid or fatty alcohol, and a surfactant. Also, the present invention is concerned with a method of preparing such compositions.

### BACKGROUND ART

Itraconazole is an azole antifungal agent having a molecular formula of C₃₅H₃₀C₁₂N₈O₄ and a molecular weight of 705.64. Itraconazole, which exists in the form of a light yellow powder, is poorly soluble in water and slightly soluble in alcohol, showing solubility of ≦1 μg/ml and 300 μg/ml, respectively, while being easily soluble in methylene chloride, showing solubility of 239 mg/ml. Also, itraconazole, as a weak basic drug (pKₐ=3.7), is almost fully ionized at low pH, such as in gastric juices, and has high fat solubility. When being orally administered, parenterally and topically, itraconazole is known to show a wide-ranging antifungal activity(U.S. Pat. No. 4,267,179). As disclosed in U.S. Pat. No. 4,267,179, itraconazole or (±)-cis-4-[4-[4-[4-[[2-(2,4-dichlorophenyl)-2-(1H-1,2,4-triazol-1-ylmethyloxolan-4-yl]methoxy]phenyl]-1-piperazinyl]phenyl]-2,4-dihydro-2-(1-methylpropyl)-3H-1,2,4-triazol-3-one, is a broad spectrum antifungal compound developed for oral, parenteral and topical use. Also, International Pat. No. WO 93/19061 discloses a method of preparing itraconazole consisting of a mixture of four diastereoisomers, and use of itraconazole.

However, when being administered to the body, a large number of drugs containing itraconazole are problematic in terms of being low in solubility and dissolution rate in digestive fluids owing to poor water solublility of itraconazole, thus reducing its bioavailability. That is, drugs in a solid form can be absorbed through endothelial cells when being dissolved in gastrointestinal juice. Therefore, in case of poorly water-soluble drugs, because their dissolution rate from their solid preparations is slow in gastrointestinal juice, their dissolution process is a rate-limiting step for their absorption. Accordingly, the dissolution rate of drugs directly affects the time required to exert their effects, as well as strength and duration of their efficacy. That is, because concentration of drugs in blood is a function of their absorption rate and degradation rate, their poor dissolution results in reduced maximum concentration in blood as well as changes in duration of their effective concentration in blood. To resolve these problems of the poorly water-soluble drugs such as itraconazole, there have been a variety of attempts to increase their solubility or dissolution rate and thus improve their bioavailability. However, poorly water-soluble itraconazole has many limitations in being formulated into an economical and pharmaceutically acceptable form, while enhancing its solubility and bioavailability.

To resolve these problems, a variety of pharmaceutical formulations have been developed with an aim to increase solubility or dissolution rate of poorly water-soluble drugs in the field of pharmaceutics. For example, in order to improve their bioavailability, there are many reports related with micronization with which particle diameter can be regulated, polymorphism and amorphous powder preparations, eutectic mixture preparations, micelle formation using surfactants, a solvent deposition method, a dry elixir method, a spray-drying method, a co-precipitation method using an inert water soluble carrier, a solid dispersion method, an inclusion complex preparation using cyclodextrins, suitable solvents and drugs capable of being used together, additives, and the like. Despite these efforts, these methods still have problems in terms of economy and efficiency because solubility of drugs varies depending on a method of preparing their pharmaceutical formulation. Pharmaceutical preparations of drugs using the solid dispersion method are disclosed in the following references.

(1) International Pat. No. WO 85/02767 and U.S. Pat. No. 4,764,604 disclose a complex increased in solubility and bioavailability of drugs, which is prepared using cyclodextrin or its derivatives.

(2) International Pat. No. WO 90/11754 discloses an aerosol preparation containing drugs having reduced particle size, thus facilitating administration of drugs.

(3) International Pat. No. WO 93/15719 discloses an externally applied liposome preparation containing itraconazole, which is prepared using phospholipid and a solvent system.

(4) International Pat. No. WO 95/31178 discloses an externally applied preparation using an emulsion or a liquid solution prepared using cyclodextrin or its derivatives, which is attachable in the nasal mucous membrane or the vaginal mucous membrane.

(5) International Pat. No. WO 94/05236 discloses an orally administrable formulation improved in solubility and bioavailability of drugs, in which a bead having a 25-30 mesh sugar sphere as a core material is coated with a hydrophilic polymer, in particular hydroxypropyl methylcellulose, and an antifungal agent, in particular itraconazole, finished with a sealing film coat, and filled into capsules suitable for oral administration, and its pharmaceutical preparation containing itraconazole is now commercially available, and is known as “Sporanox™”.

(6) International Pat. No. WO 97/44014 discloses pharmaceutical compositions of itraconazole comprising particles obtainable by first preparing a solid dispersion comprising itraconazole and an appropriate water-soluble polymer, and then optionally grinding or milling the solid dispersion through various techniques including a melt-extrusion process, which has improved bioavailability by increasing dissolution rate of drugs, as well as reducing variance in bioavailability according to food intake.

As described in International Pat. No. WO 94/05236 (Janssen Pharmaceutica N. V.), beads with good solubility and bioavailability can be prepared by spraying a mixture of itraconazole and a hydrophilic polymer, more particularly hydroxypropyl methylcellulose onto a sugar sphere of very small core of about 25-30 mesh, followed by drying and sealing with polyethyleneglycole. About 460 mg of the beads, equivalent to about 100 mg of itraconazole, is filled into a capsule suitable for oral administration, and two of these capsules are administered once daily to a patient suffering from a fungal infection. However, the beads have disadvantages as follows. Their bioavailability is easily influenced by food intake, and their preparation process is complicated. An organic solvent such as methylene chloride is employed, which is harmful to human beings by showing residual toxicity. In addition, there is a large difference in their bioavailability among individuals.

On the other hand, as disclosed in International Pat. No. WO 97/44014, pharmaceutical compositions comprising particles of a particle size of from 50 to 500 μm, which is obtainable by preparing a solid dispersion through a process of vacuum-melting a mixture containing intraconazole and an appropriate water-soluble polymer, cooling the melted mixture to solidify it, and then optionally grinding or milling the solid dispersion, where the solid dispersion can be also prepared by spray-drying the extrudate, or simply pouring the extrude onto a wide surface and then evaporating the solvent. Such a single dosage form can be administered once daily and further comprise pharmaceutically acceptable additives. However, this method has drawbacks in that even though itraconazole is thermally very stable, the water-soluble polymer and additives can be carbonized or denatured because the melt-extruder is operated at high temperature between about 120° C. and about 300° C., and control of high temperature is difficult and complicated, thus reproductivity is poor and high cost are incurred.

Korean Pat. Applicationn No. 1998/27730 (Choongwae Pharma. Corp.) discloses an orally administrable formulation of poorly water-soluble itraconazole, in which itraconazole and a pH-dependent water-soluble polymer, which is pharmaceutically stable and rapidly dissolved in low pH and rapidly dissolved, are dissolved in a solvent and dispersed, and the mixture is then spray-dried to give a solid dispersion, resulting in increased solubility of itraconazole and its rapid dissolution regardless of food intake, and thus improved bioavailability of itraconazole, wherein the solid dispersion means a homogeneous dispersion of a polymer in a solid state or one or more active ingredients in an inert carrier. However, including lyophilization, natural-drying and drying under nitrogen gas, the solvent methods by which a solid dispersion is prepared using a water-soluble polymer as a carrier is problematic in terms of generally giving low reproducibility of efficiency of pharmaceutical preparations, as well as incurring high costs and requiring a long time to prepare such pharmaceutical preparations. When using the vacuum-melting method, stability of drugs can be influenced because drugs and carriers should be vacuum-melted at temperatures more than their melting points, and a process of preparing pharmaceutical preparations must be performed with caution because the cooling condition of the extrudate negatively affects efficacy of pharmaceutical preparations. In addition, a solvent-melting method, which is utilized when the solvent method or the vacuum-melting method are not allowed to be used alone, has a disadvantage in that it takes a long time to prepare the pharmaceutical preparations. Especially, employed organic solvents are harmful to the human body owing to their residual properties, and the solid dispersion particles are easily aggregated and thus hard to recrystallize. Further, reduction of their dosage obtainable by increasing dissolution rate of drugs is not achieved. Dissolution rate of drugs is high in artificial gastric juice (pH 1.2), but there is no reliable data for bioavialability of the pharmaceutical preparations in the human body.

As disclosed in Korean Pat. Application No. 1997/70873 (Dong-A Pharmaceutical Co. Ltd.), a novel pharmaceutical composition with improved solubility and dissolution rate can be prepared by mixing poorly water-soluble itraconazole with a pharmaceutically acceptable water-soluble sugar (sucrose, glucose, lactose, mannitol, sorbitol, fructose, etc.), heating and vacuum-melting the mixture, and then cooling the melted mixture, which is formulated into capsules or tablets. The pharmaceutical composition containing itraconazole has excellent stability and is economical thanks to the use of inexpensive sugars and simplicity of the preparation process. Such a method does not employ an organic solvent, but has a disadvantage in that the vacuum-melting step is performed at about 160-180° C. at which the water-soluble sugar can be denatured, resulting in high-priced products, in addition that reduction of dosage obtainable by improving dissolution rate of the drug is not achieved. Moreover, there is no detailed information for bioavailability in human beings, thus making its practical use difficult.

As described above, with the aim of achieving improved solubility and dissolution rate of poorly water-soluble itraconazole, the solid dispersion containing itraconazole can be prepared using various techniques including vacuum melting-extrusion, spray-drying and solution-evaporation, but all such techniques have obvious drawbacks of being inefficient, complicated, non-economical and harmful owing to the use of organic solvents.

### DISCLOSURE OF INVENTION

Leading to the present invention, the intensive and thorough research into a pharmaceutical composition containing itraconazole, conducted by the present inventors, with the aim of solving the problems in dissolution, absorption by the human body, and pharmaceutical formulation of itraconazole that are encountered in the background art, resulted in the finding that a viscous composition comprising itraconazole, fatty acid or fatty alcohol, and a surfactant is rapidly dissolved and dispersed in the gastrointestinal juice and forms a very stable microemulsion, thus improving bioavaiability of itraconazole by increasing its dissolution rate.

It is therefore an object of the present invention to provide a novel composition containing itraconazole with remarkably improved dissolution and bioavailability.

It is another object of the present invention to provide a viscous composition comprising itraconazole, fatty acid or fatty alcohol, and a surfactant.

It is still another object of the present invention to provide a viscous composition prepared by melting or dispersing poorly water-soluble itraconazole in fatty acid, a surfactant and a pharmaceutically acceptable additives.

It is a further object of the present invention to provide a soft capsule or hard capsule preparation filled with the said composition.

It is a still further object of the present invention to provide a solid powdery preparation prepared by mixing the composition with a base and then drying the mixture.

It is a still further object of the present invention to provide a method of preparing the said composition.

It is a still further object of the present invention to provide a method of preparing the said composition, comprising the steps of melt-mixing itraconazole, fatty acid, or fatty alcohol, a surfactant and a pharmaceutically mixable additive, and milling the resulting mixture.

### BRIEF DESCRIPTION OF DRAWINGS

FIG. 1 is a graph in which plasma concentrations (μg/ml) of itraconazole are plotted against time after oral administration of a soft capsule containing 40 mg of itraconazole according to the present invention and a commercially available preparation (Sporanox™ capsule) containing 100 mg of itraconazole.

### BEST MODE FOR CARRYING OUT THE INVENTION

The present invention is related to a composition containing itraconazole with significantly improved bioavialability and a method of preparing the same. In accordance with the present invention, there is provided a composition comprising (a) poorly water-soluble itraconazole, (b) fatty acid or fatty alcohol and (c) a surfactant, and a preparation method thereof. In accordance with the present invention, the composition is in a viscous form in which poorly water-soluble itraconazole is dissolved or dispersed in fatty acid and the surfactant. Also, the composition is dissolved in the water to form a microemulsion, thereby allowing its use in a self-microemulsifying drug delivery system (SMEDDS).

Examples of fatty acid or fatty alcohol useful in the composition of the present invention include, but are not limited to, oleic acid, stearyl alcohol, myristic acid, linoleic acid or lauric acid, capric acid, caprylic acid, and caproic acid. Preferred are oleic acid, lauric acid, capric acid, caprylic acid and caproic acid.

Examples of the surfactant useful in the composition of the present invention include, but are not limited to, sodium lauryl sulfate and its derivatives, poloxamer and its derivatives, saturated polyglycolized glyceride (so-called Gelucire), labrasol, various polysorbates, which are exemplified as polyoxyethylene sorbitan monolaurate (hereinafter referred to as “Tween 20”), polyoxyethylene sorbitan monopalmitate (hereinafter referred to as “Tween 40”), polyoxyethylene sorbitan monostearate (hereinafter referred to as “Tween 60”) and polyoxyethylene sorbitan monooleate (hereinafter referred to as “Tween 80”), sorbitan esters, which are exemplified as sorbitan monolaurate (hereinafter referred to as “Span 20”), sorbitan monopalmitate (hereinafter referred to as “Span 40”), sorbitan monostearate (hereinafter referred to as “Span 60”), sorbitan monooleate (hereinafter referred to as “Span 80”), sorbitan trilaurate (hereinafter referred to as “Span 25”) sorbitan trioleate (hereinafter referred to as “Span 85”) and sorbitan tristearate (hereinafter referred to as “Span 65”), cremophor, PEG-60 hydrogenated castor oil, PEG-40 hydrogenated castor oil, sodium lauryl glutamate, and disodium cocoamphodiacetate. Preferred are sodium lauryl sulfate which is an anionic surfactant and its derivatives, Tween 20, 40, 60 and 80 which are non-ionic surfactants, and Span 20, 40, 60, 80, 25, 85 and 65 which are sorbitan esters, and most preferred are Tween 20, 40, 60 and 80.

It is preferable that the composition according to the present invention further comprises one or more organic acids to prevent recrystallization of itraconazole during storage. Examples of the organic acids useful in the present invention include citric acid. In addition, the said organic acids include fumaric acid, maleic acid, malic acid, salicylic acid, formic acid, glycolic acid, lactic acid, acetic acid, propionic acid, and α- or β-hydroxy acid.

The composition according to the present invention further comprises a cosurfactant along with the surfactant to effectively stabilize the viscous self-microemulsifying drug delivery system (SMEDDS) and increase dissolution. Examples of the cosurfactant useful in the present invention include polyethylene glycol (PEG) and its derivatives, ethanol-containing alcohols, transcutol (for example, ethoxy diglycol), propylene glycol, ethyl oleate, methyl pyrrolidone, ethyl pyrrolidone, propyl pyrrolidinone, glycerol, xylitol, sorbitol, dextrose, and mannitol. Preferred cosurfactants are transcutol, propylene glycol, and ethyl oleate.

In addition, the composition according to the present invention additionally comprises various additives in a range not negatively affecting state and efficacy thereof, which are exemplified as oils, anti-oxidants, disintegrants and foaming agents.

Examples of oils useful in the composition of the present invention include, but are not limited to, various labrafac (for example, caprylic/capric triglyceride or medium-chain triglyceride), propylene glycol caprylate/caprate, various labrafil (for example, oleoil microgol-6 glyceride, linoleoil microgol-6 glyceride), propyleneglycol laurate (so-called lauroglycol), glyceryl monoleate, glyceryl monolinoleate, glyceryl monoleate/linoleate, α-bisabolol, tocopheryl acetate, liposome, phospholipid including phosphatidylcholine, di-C₁₂₋₁₃ alkyl malate, coco-caprylate/caprate, cetyl octanoate, and hydrogenated castor oil.

Examples of the anti-oxidants useful in the composition of the present invention include, but are not limited to, butylated hydroxytoluene (BHT), sodium bisulfite, α-tocopherol, vitamin C, β-carotene, ascobylpalmitate, tocopherol acetate, fumaric acid, nalic acid, butylated hydroxyanisole, propyl gallate, and sodium ascorbate. Such anti-oxidants can be added directly to the viscous composition or during the process for preparing the solid preparation, in a range of 0.0001-10% of total amount of the composition.

Examples of the disintegrants useful in the composition of the present invention include, but are not limited to, croscarmellose sodium, sodium starch glycolate (Primojel), microcrystalline cellulose (Avicel), crospovidone (Polyplasdone) and other commercially available PVP, low-substituted hydroxypropylcellulose, alginic acid, calcium salts and potassium salts of carboxy methyl cellulose (CMC), colloidal silicon dioxide, guar gum, magnesium aluminum silicate, methylcellulose, powdered cellulose, starch and sodium alginate. The disintegrants may be added directly to the composition of the viscous invention or to the solid powdery preparation thereof using a pharmaceutically acceptable method when being formulated into compressed particles, pellets, granules or tablets. The range of used amount is typically in an amount of 1-50% by weight.

Examples of the foaming agents useful in the composition of the present invention include, but are not limited to, NaHCO₃ and Na₂CO₃.

The composition according to the present invention may be administrated to various preparations. For example, the composition can be administrated into capsules prepared by filling into capsules comprising soft or hard capsules, or compressed particles, pellet or tablets by melting in a mixture with a base and then dry-powdered. Examples of the base useful in the solid powder process include, but are not limited to, various polymeric bases which are exemplified as polyethylene glycol (PEG), carbowax, saturated polyglycolized glyceride (so-called Gelucire), methyl cellulose, ethyl cellulose, hydroxypropylmethylcellulose, carboxymethylcellulose, glycerolmonostearate, or polyvinyl pyrrolidone (PVP). Also, the composition according to the present invention further includes one or more water-soluble polymeric bases, which are exemplified as gelatin, gums, carbohydrates, cellulose and its derivatives, polyethylene oxide and its derivatives, polyvinyl alcohol, polyacrylic acid and its derivatives, polymethylacrylate, and inorganic compounds.

The composition according to the present invention is orally administrated to human beings. When being orally administered, the composition according to the present invention has 2-4 times higher bioavailability of itraconazole than that of the commercially available preparation (Sporanox™, Janssen Pharmaceutica N. V.), in which the composition of the present invention containing a small amount of about 30-70 mg itraconazole has efficacy equivalent to that of the commercially available preparation (Sporanox™, Janssen Pharmaceutica N. V.) containing 100 mg itraconazole. An amount of itraconazole to be contained in the composition may be properly determined depending on a patient's age, sex, disease state and the like, typically 30-120 mg, preferably 30-80 mg, more preferably 30-70 mg, and most preferably 40-60 mg.

In addition, in accordance with the present invention, there is provided a method of preparing the said composition, which is prepared by mixing itraconazole, fatty acid and a surfactant, additionally including organic acid, oil, an anti-oxidant, a disintegrant and a foaming agent according to intended use, and heat-melting or vacuum-melting and then cooling the mixture, including an additional step of milling the mixture according to intended use. In detail, the method comprises the steps of (1) adding itraconazole to a mixture of fatty acid and a surfactant, additionally including organic acid, oil, an anti-oxidant, a disintegrant and a foaming agent according to intended use, and heat-melting or vacuum-melting and then cooling the mixture, to form a transparent viscous composition to be used Self-microemulsifying drug delivery system and (2) preferably further comprises a step of roll-milling (most preferably, 3-roll milling) the resulting viscous semi-solid composition. When itraconazole is dispersed through the heat-melting or vacuum-milling step and thus a compositon in a wax phase is formed, the composition can be additionally roll-milled to give a more homogeneously dispersed viscous composition. In addition, the method (3) further comprises the steps of heat-melting or vacuum-melting and cooling a part of the said mixture, first roll-milling the said part, and to form the viscous composition, additionally roll-milling the said part to which the rest of the mixture is added. Consequently, itraconazole contained in the said composition is increased in dissolution rate and thus improved in bioavailability (see, Experimental Examples 1 to 4, in which features of the method according to the present invention are described in more detail).

In accordance with an experimental example of the present invention, a viscous semi-solid composition with improved dissolution property and bioavailability can be prepared by heat-melting or vacuum-melting and cooling a mixture containing itraconazole in an amount of 8-12 parts by weight, fatty acid in an amount of 8-60 parts by weight, preferably 8-48 parts by weight, and most preferably 8-12 parts by weight, the surfactant in an amount of 64-120 parts by weight, preferably 80-120 parts by weight, and organic acid in an amount of 16-24 parts by weight, resulting in forming a light brown viscous semi-solid composition, itself or optionally by roll-milling step. In addition, when one or more additives selected from the group consisting of oil, an anti-oxidant, a disintegrant and a foaming agent are added thereto in a small amount, the state and dissolution rate of the viscous composition is not changed. In detail, a viscous semi-solid composition with improved dissolution property and bioavailability is prepared by heat-melting or vacuum-melting and cooling a mixture containing itraconazole in an amount of 8-12 parts by weight, oleic acid in an amount of 8-60 parts by weight, preferably 8-48 parts by weight, and most preferably 8-12 parts by weight, Tween 20 or 80 in an amount of 64-120 parts by weight, preferably 80-120 parts by weight, and citric acid in an amount of 16-24 parts by weight, and optionally roll-milling the resulting viscous semi-solid composition.

In accordance with another experimental example of the present invention, a viscous semi-solid composition with improved dissolution property and bioavailability can be prepared by heat-melting or vacuum-melting and cooling a mixture containing itraconazole in an amount of 8-12 parts by weight, fatty acid selected from the group consisting of oleic acid, lauric acid, caprylic acid and mixtures thereof in an amount of 40-60 parts by weight, preferably a mixture of laulic acid and caprylic acid in an amount of 40-60 parts by weight, and most preferably lauric acid in an amount of 8-12 parts by weight and caprylic acid in an amount of 32-48 parts by weight, Tween 20 or 80 in an amount of 64-96 parts by weight, and citric acid in an amount of 16-24 parts by weight, and optionally roll milling the resulting viscous semi-solid composition. In addition, when one or more additives selected from the group consisting of oil, an anti-oxidant, a disintegrant and a foaming agent are added thereto in a small amount, the state and dissolution rate of the viscous composition is not changed.

The present invention will be explained in more detail with reference to the following examples. However, the following examples are provided only to illustrate the present invention, and the present invention is not limited to them. Therefore, it will be apparent to one skilled in the art that various changes and modifications can be made therein without departing from the spirit and scope thereof.

### EXAMPLE 1

According to the same method as in Experimental Example 1, a mixture containing 1 g of itraconazole, 3 g of oleic acid and 3 g of Tween 80 was heat-melted or vacuum-melted. Thereafter, the mixture was cooled and roll-milled to form a viscous SMEDDS.

### EXAMPLE 2

According to the same method as in Experimental Example 1, a mixture containing 1 g of itraconazole, 3 g of oleic acid, 1.5 g of Tween 80 and 1.5 g of Tween 20 was heat-melted or vacuum-melted. Thereafter, the mixture was cooled and roll-milled to form a viscous SMEDDS.

### EXAMPLE 3

According to the same method as in Experimental Example 1, a mixture containing 1 g of itraconazole, 3 g of oleic acid and 3 g of Tween 20 was heat-melted or vacuum-melted. Thereafter, the mixture was cooled and roll-milled to form a viscous SMEDDS.

### EXAMPLE 4

According to the same method as in Experimental Example 1, a mixture containing 1 g of itraconazole, 3 g of oleic acid and 6 g of Tween 80 was heat-melted or vacuum-melted. Thereafter, the mixture was cooled and roll-milled to form a viscous SMEDDS.

### EXAMPLE 5

According to the same method as in Experimental Example 1, a mixture containing 1 g of itraconazole, 3 g of oleic acid and 3 g of Tween 80 was heat-melted or vacuum-melted. Thereafter, the mixture was cooled and roll-milled. 1 g of 1-ethyl-2-pyrrolidinone was mixed to the mixture to form a viscous SMEDDS.

### EXAMPLE 6

According to the same method as in Experimental Example 1, a mixture containing 1 g of itraconazole, 1 g of oleic acid and 1 g of Tween 80 was heat-melted or vacuum-melted. Thereafter the resulting mixture was cooled and roll-milled. 1 g of 1-ethyl-2-pyrrolidinone was mixed to the resulting mixture to form a viscous SMEDDS.

### EXAMPLE 7

According to the same method as in Experimental Example 1, a mixture containing 1 g of itraconazole, 1 g of oleic acid and 1 g of Tween 80 was heat-melted or vacuum-melted. Thereafter the resulting mixture was cooled and roll-milled. 1 g of 1-methyl-2-pyrrolidinone was added to the resulting mixture, and homogeneously mixed to form a viscous SMEDDS.

### EXAMPLE 8

According to the same method as in Experimental Example 1, a mixture containing 1 g of itraconazole, 3 g of oleic acid and 6 g of Tween 80 was heat-melted or vacuum-melted. Thereafter the resulting mixture was cooled and roll-milled. 1 g of carmellose sodium was added to the resulting mixture, and homogeneously mixed to form a viscous SMEDDS.

### EXAMPLE 9

According to the same method as in Experimental Example 1, a mixture containing 1 g of itraconazole, 3 g of caproic acid and 16 g of Tween 80 was heat-melted or vacuum-melted and then cooled. The resulting mixture was roll-milled to form a viscous SMEDDS.

### EXAMPLE 10

According to the same method as in Experimental Example 1, a mixture containing 1 g of itraconazole, 3 g of caprylic acid and 16 g of Tween 80 was heat-melted or vacuum-melted and then cooled. The resulting mixture was roll-milled to form a viscous SMEDDS.

### EXAMPLE 11

According to the same method as in Experimental Example 1, a mixture containing 1 g of itraconazole, 3 g of capric acid and 16 g of Tween 80 was heat-melted or vacuum-melted and then cooled. The resulting mixture was roll-milled to form a viscous SMEDDS.

### EXAMPLE 12

According to the same method as in Experimental Example 1, a mixture containing 1 g of itraconazole, 3 g of lauric acid and 16 g of Tween 80 was heat-melted or vacuum-melted and then cooled. The resulting mixture was roll-milled to form a viscous SMEDDS.

### EXAMPLE 13

According to the same method as in Experimental Example 1, a mixture containing 1 g of itraconazole, 3 g of myristic acid and 16 g of Tween 80 was heat-melted or vacuum-melted and then cooled. The resulting mixture was roll-milled to form a viscous SMEDDS.

### EXAMPLE 14

According to the same method as in Experimental Example 1, a mixture containing 1 g of itraconazole, 3 g of palmitic acid and 16 g of Tween 80 was heat-melted or vacuum-melted and then cooled. The resulting mixture was roll-milled to form a viscous SMEDDS.

### EXAMPLE 15

According to the same method as in Experimental Example 1, a mixture containing 1 g of itraconazole, 3 g of stearic acid and 16 g of Tween 80 was heat-melted or vacuum-melted and then cooled. The resulting mixture was roll-milled to form a viscous SMEDDS.

### EXAMPLE 16

According to the same method as in Experimental Example 1, a mixture containing 1 g of itraconazole, 3 g of linoleic acid and 16 g of Tween 80 was heat-melted or vacuum-melted and then cooled. The resulting mixture was roll-milled to form a viscous SMEDDS.

### EXAMPLE 17

According to the same method as in Experimental Example 1, a mixture containing 1 g of itraconazole, 3 g of oleil alchol and 16 g of Tween 80 was heat-melted or vacuum-melted and then cooled. The resulting mixture was roll-milled to form a viscous SMEDDS.

### EXAMPLE 18

According to the same method as in Experimental Example 1, a mixture containing 1 g of itraconazole, 3 g of cetyl alchol and 16 g of Tween 80 was heat-melted or vacuum-melted and then cooled. The resulting mixture was roll-milled to form a viscous SMEDDS.

### EXAMPLE 19

According to the same method as in Experimental Example 1, a mixture containing 1 g of itraconazole, 3 g of oleic acid and 6 g of Tween 80 was heat-melted or vacuum-melted and then cooled and roll-milled. 0.3 g of croscarmellose sodium and 1 g of ethoxy diglycol were added to the cooled mixture, and homogeneously mixed, and the resulting mixture was roll-milled to form a viscous SMEDDS.

### EXAMPLE 20

According to the same method as in Experimental Example 1, a mixture containing 1 g of itraconazole, 3 g of oleic acid and 6 g of Tween 80 was heat-melted or vacuum-melted and then cooled and roll-milled. 0.3 g of croscarmellose sodium and 3 g of ethoxy diglycol were added to the cooled mixture, and homogeneously mixed, and the resulting mixture was roll-milled to form a viscous SMEDDS.

### EXAMPLE 21

According to the same method as in Experimental Example 1, a mixture containing 1 g of itraconazole, 3 g of oleic acid and 6 g of Tween 80 was heat-melted or vacuum-melted and then cooled and roll-milled. 0.3 g of croscarmellose sodium and 5 g of ethoxy diglycol were added to the cooled mixture, and homogeneously mixed, and the resulting mixture was roll-milled to form a viscous SMEDDS.

### EXAMPLE 22

According to the same method as in Experimental Example 2, a mixture containing 1 g of itraconazole, 3 g of oleic acid and 6 g of Tween 80 was heat-melted or vacuum-melted and then cooled and roll-milled. 10 g of Tween 20 and 0.3 g of croscarmellose sodium were added to the cooled mixture, and homogeneously mixed, and the resulting mixture was roll-milled to form a viscous SMEDDS.

### EXAMPLE 23

According to the same method as in Experimental Example 2, a mixture containing 1 g of itraconazole, 3 g of oleic acid and 6 g of Tween 80 was heat-melted or vacuum-melted and then cooled and roll-milled. 10 g of Tween 80 and 0.4 g of croscarmellose sodium were added to the cooled mixture, and homogeneously mixed, and the resulting mixture was roll-milled to form a viscous SMEDDS.

### EXAMPLE 24

According to the same method as in Experimental Example 2, a mixture containing 1 g of itraconazole, 3 g of oleic acid and 6 g of Tween 80 was heat-melted or vacuum-melted and then cooled and roll-milled. 10 g of Tween 80 and 0.3 g of croscarmellose sodium were added to the cooled mixture, and homogeneously mixed, and the resulting mixture was roll-milled to form a viscous SMEDDS.

### EXAMPLE 25

According to the same method as in Experimental Example 2, a mixture containing 1 g of itraconazole, 2 g of oleic acid and 7 g of Tween 80 was heat-melted or vacuum-melted and then cooled and roll-milled. 10 g of Tween 80 and 0.4 g of croscarmellose sodium were added to the cooled mixture, and homogeneously mixed, and the resulting mixture was roll-milled to form a viscous SMEDDS.

### EXAMPLE 26

According to the same method as in Experimental Example 2, a mixture containing 1 g of itraconazole, 2 g of oleic acid and 6 g of Tween 80 was heat-melted or vacuum-melted and then cooled and roll-milled. 3 g of Tween 80, 0.5 g of croscarmellose sodium and 1 g of ethoxy diglycol were added to the cooled mixture, and homogeneously mixed, and the resulting mixture was roll-milled to form a viscous SMEDDS.

### EXAMPLE 27

According to the same method as in Experimental Example 2, a mixture containing 1 g of itraconazole, 2 g of oleic acid and 6 g of Tween 80 was heat-melted or vacuum-melted and then cooled and roll-milled. 5 g of Tween 80, 0.5 g of croscarmellose sodium and 1 g of ethoxy diglycol were added to the cooled mixture, and homogeneously mixed, and the resulting mixture was roll-milled to form a viscous SMEDDS.

### EXAMPLE 28

According to the same method as in Experimental Example 2, a mixture containing 1 g of itraconazole, 2 g of oleic acid and 6 g of Tween 80 was heat-melted or vacuum-melted and then cooled and roll-milled. 9 g of Tween 80, 0.5 g of croscarmellose sodium and 1 g of ethoxy diglycol were added to the cooled mixture, and homogeneously mixed, and the resulting mixture was roll-milled to form a viscous SMEDDS.

### EXAMPLE 29

According to the same method as in Experimental Example 3, a mixture containing 1 g of itraconazole, 2 g of citric acid and 4 g of Tween 80 was heat-melted or vacuum-melted and then cooled to 40° C. 4 g of Tween 80 and 1 g of laulic acid were added to the cooled mixture, and homogeneously mixed, and the resulting mixture was cooled at room temperature to form a viscous SMEDDS.

### EXAMPLE 30

According to the same method as in Experimental Example 3, a mixture containing 1 g of itraconazole, 2 g of citric acid and 4 g of Tween 80 was heat-melted or vacuum-melted and then cooled to 40° C. 4 g of Tween 80, 0.5 g of caprylic acid and 1 g of laulic acid were added to the cooled mixture, and homogeneously mixed, and the resulting mixture was cooled at room temperature to form a viscous SMEDDS.

### EXAMPLE 31

According to the same method as in Experimental Example 3, a mixture containing 1 g of itraconazole, 2 g of citric acid and 4 g of Tween 80 was heat-melted or vacuum-melted and then cooled to 40° C. 4 g of Tween 80, 1 g of caprylic acid and 0.5 g of laulic acid were added to the cooled mixture, and homogeneously mixed, and the resulting mixture was cooled at room temperature to form a viscous SMEDDS.

### EXAMPLE 32

According to the same method as in Experimental Example 3, a mixture containing 1 g of itraconazole, 2 g of citric acid and 4 g of Tween 80 was heat-melted or vacuum-melted and then cooled to 40° C. 4 g of Tween 80, 1 g of caprylic acid and 1 g of laulic acid were added to the cooled mixture, and homogeneously mixed, and the resulting mixture was cooled at room temperature to form a viscous SMEDDS.

### EXAMPLE 33

According to the same method as in Experimental Example 3, a mixture containing 1 g of itraconazole, 2 g of citric acid and 4 g of Tween 80 was heat-melted or vacuum-melted and then cooled to 40° C. 4 g of Tween 80, 0.5 g of caprylic acid and 0.5 g of laulic acid were added to the cooled mixture, and homogeneously mixed, and the resulting mixture was cooled at room temperature to form a viscous SMEDDS.

### EXAMPLE 34

According to the same method as in Experimental Example 3, a mixture containing 1 g of itraconazole, 2 g of citric acid and 4 g of Tween 80 was heat-melted or vacuum-melted and then cooled to 40° C. 4 g of Tween 80, 0.7 g of caprylic acid and 0.3 g of laulic acid were added to the cooled mixture, and homogeneously mixed, and the resulting mixture was cooled at room temperature to form a viscous SMEDDS.

### EXAMPLE 35

According to the same method as in Experimental Example 3, a mixture containing 1 g of itraconazole, 2 g of citric acid and 4 g of Tween 80 was heat-melted or vacuum-melted and then cooled to 40° C. 4 g of Tween 80, 0.6 g of caprylic acid and 0.4 g of laulic acid were added to the cooled mixture, and homogeneously mixed, and the resulting mixture was cooled at room temperature to form a viscous SMEDDS.

### EXAMPLE 36

According to the same method as in Experimental Example 3, a mixture containing 1 g of itraconazole, 2 g of citric acid and 4 g of Tween 80 was heat-melted or vacuum-melted and then cooled to 40° C. 4 g of Tween 80, 0.8 g of caprylic acid and 0.4 g of laulic acid were added to the cooled mixture, and homogeneously mixed, and the resulting mixture was cooled at room temperature to form a viscous SMEDDS.

### EXAMPLE 37

According to the same method as in Experimental Example 3, a mixture containing 1 g of itraconazole, 2 g of citric acid and 4 g of Tween 80 was heat-melted or vacuum-melted and then cooled to 40° C. 4 g of Tween 80 and 1 g of caprylic acid were added to the cooled mixture, and homogeneously mixed, and the resulting mixture was cooled at room temperature to form a viscous SMEDDS.

### EXAMPLE 38

According to the same method as in Experimental Example 3, a mixture containing 1 g of itraconazole, 2 g of citric acid and 4 g of Tween 80 was heat-melted or vacuum-melted and then cooled to 40° C. 4 g of Tween 80, 0.75g of caprylic acid and 0.5 g of laulic acid were added to the cooled mixture, and homogeneously mixed, and the resulting mixture was cooled at room temperature to form a viscous SMEDDS.

### EXAMPLE 39

According to the same method as in Experimental Example 3, a mixture containing 1 g of itraconazole, 2 g of citric acid and 4 g of Tween 20 was heat-melted or vacuum-melted and then cooled to 40° C. 4 g of Tween 20, 3 g of caprylic acid and 1 g of laulic acid were added to the cooled mixture, and homogeneously mixed, and the resulting mixture was cooled at room temperature to form a viscous SMEDDS.

### EXAMPLE 40

According to the same method as in Experimental Example 3, a mixture containing 1 g of itraconazole, 2 g of citric acid and 4 g of Tween 20 was heat-melted or vacuum-melted and then cooled to 40° C. 4 g of Tween 20, 4 g of caprylic acid and 1 g of laulic acid were added to the cooled mixture, and homogeneously mixed, and the resulting mixture was cooled at room temperature to form a viscous SMEDDS.

### EXAMPLE 41

According to the same method as in Experimental Example 3, a mixture containing 1 g of itraconazole, 2 g of citric acid and 4 g of Tween 20 was heat-melted or vacuum-melted and then cooled to 40° C. 4 g of Tween 20, 0.7g of caprylic acid and 0.3 g of laulic acid were added to the cooled mixture, and homogeneously mixed, and the resulting mixture was cooled at room temperature to form a viscous SMEDDS.

### EXAMPLE 42

According to the same method as in Experimental Example 3, a mixture containing 1 g of itraconazole, 2 g of citric acid and 4 g of Tween 20 was heat-melted or vacuum-melted and then cooled to 40° C. 4 g of Tween 20, 0.6 g of caprylic acid and 0.4 g of laulic acid were added to the cooled mixture, and homogeneously mixed, and the resulting mixture was cooled at room temperature to form a viscous SMEDDS.

### EXAMPLE 43

According to the same method as in Experimental Example 3, a mixture containing 1 g of itraconazole, 2 g of citric acid and 4 g of Tween 20 was heat-melted or vacuum-melted and then cooled to 40° C. 4 g of Tween 20, 0.75g of caprylic acid and 0.5 g of laulic acid were added to the cooled mixture, and homogeneously mixed, and the resulting mixture was cooled at room temperature to form a viscous SMEDDS.

### EXAMPLE 44

According to the same method as in Experimental Example 3, a mixture containing 1 g of itraconazole, 2 g of citric acid and 4 g of Tween 20 was heat-melted or vacuum-melted and then cooled to 40° C. 4 g of Tween 20, 0.5 g of caprylic acid and 0.5 g of laulic acid were added to the cooled mixture, and homogeneously mixed, and the resulting mixture was cooled at room temperature to form a viscous SMEDDS.

### EXAMPLE 45

According to the same method as in Experimental Example 3, a mixture containing 1 g of itraconazole, 2 g of citric acid and 4 g of Tween 20 was heat-melted or vacuum-melted and then cooled to 40° C. 4 g of Tween 20, 0.5 g of caprylic acid and 0.5 g of oleic acid were added to the cooled mixture, and homogeneously mixed, and the resulting mixture was cooled at room temperature to form a viscous SMEDDS.

### EXAMPLE 46

According to the same method as in Experimental Example 4, a mixture containing 1 g of itraconazole, 2 g of citric acid and 4 g of Tween 80 was heat-melted or vacuum-melted and then cooled to 40° C. 4 g of Tween 80 and 1 g of oleic acid were added to the cooled mixture, and homogeneously mixed, and the resulting mixture was allowed to react at room temperature for about 24 hours. Thereafter, the mixture was roll-milled to form a viscous SMEDDS.

### EXAMPLE 47

According to the same method as in Experimental Example 4, a mixture containing 1 g of itraconazole, 2 g of citric acid and 4 g of Tween 80 was heat-melted or vacuum-melted and then cooled to 40° C. 11 g of Tween 80 and 1 g of oleic acid were added to the cooled mixture, and homogeneously mixed, and the resulting mixture was allowed to react at room temperature for about 24 hours. Thereafter, the mixture was roll-milled to form a viscous SMEDDS.

### EXAMPLE 48

According to the same method as in Experimental 25 Example 4, a mixture containing 1 g of itraconazole, 2 g of citric acid and 4 g of Tween 80 was heat-melted or vacuum-melted and then cooled to 40° C. 8 g of Tween 80 and 1 g of oleic acid were added to the cooled mixture, and homogeneously mixed, and the resulting mixture was allowed to react at room temperature for about 24 hours. Thereafter, the mixture was roll-milled to form a viscous SMEDDS.

### EXAMPLE 49

According to the same method as in Experimental Example 4, a mixture containing 1 g of itraconazole, 2 g of citric acid and 4 g of Tween 80 was heat-melted or vacuum-melted and then cooled to 40° C. 10 g of Tween 80 and 1 g of oleic acid were added to the cooled mixture, and homogeneously mixed, and the resulting mixture was allowed to react at room temperature for about 24 hours. Thereafter, the mixture was roll-milled to form a viscous SMEDDS.

### EXAMPLE 50

According to the same method as in Experimental Example 4, a mixture containing 1 g of itraconazole, 2 g of citric acid and 4 g of Tween 80 was heat-melted or vacuum-melted and then cooled to 40° C. 6 g of Tween 80 and 1 g of oleic acid were added to the cooled mixture, and homogeneously mixed, and the resulting mixture was allowed to react at room temperature for about 24 hours. Thereafter, the mixture was roll-milled to form a viscous SMEDDS.

### EXAMPLE 51

According to the same method as in Experimental Example 4, a mixture containing 1 g of itraconazole, 2 g of lactic acid and 4 g of Tween 80 was heat-melted or vacuum-melted and then cooled to 40° C. 8 g of Tween 80 and 1 g of oleic acid were added to the cooled mixture, and homogeneously mixed, and the resulting mixture was allowed to react at room temperature for about 24 hours. Thereafter, the mixture was roll-milled to form a viscous SMEDDS.

### EXAMPLE 52

According to the same method as in Experimental Example 4, a mixture containing 1 g of itraconazole, 2 g of malic acid and 4 g of Tween 80 was heat-melted or 1 vacuum-melted and then cooled to 40° C. 8 g of Tween 80 and 1 g of oleic acid were added to the cooled mixture, and homogeneously mixed, and the resulting mixture was allowed to react at room temperature for about 24 hours. Thereafter, the mixture was roll-milled to form a viscous SMEDDS.

### EXAMPLE 53

According to the same method as in Experimental Example 4, a mixture containing 1 g of itraconazole, 2 g of citric acid and 4 g of Tween 80 was heat-melted or vacuum-melted and then cooled to 40° C. 6 g of Tween 80, 2 g of lactic acid and 1 g of oleic acid were added to the cooled mixture, and homogeneously mixed, and the resulting mixture was allowed to react at room temperature for about 24 hours. Thereafter, the mixture was roll-milled to form a viscous SMEDDS.

### EXAMPLE 54

According to the same method as in Experimental Example 4, a mixture containing 1 g of itraconazole, 2 g of citric acid and 4 g of Tween 80 was heat-melted or vacuum-melted and then cooled to 40° C. 6 g of Tween 80, 1 g of lactic acid and 1 g of oleic acid were added to the cooled mixture, and homogeneously mixed, and the resulting mixture was allowed to react at room temperature for about 24 hours. Thereafter, the mixture was roll-milled to form a viscous SMEDDS.

### EXAMPLE 55

According to the same method as in Experimental Example 4, a mixture containing 1 g of itraconazole, 2 g of citric acid and 4 g of Tween 80 was heat-melted or vacuum-melted and then cooled to 40° C. 5 g of Tween 80, 3 g of lactic acid and 1 g of oleic acid were added to the cooled mixture, and homogeneously mixed, and the resulting mixture was allowed to react at room temperature for about 24 hours. Thereafter, the mixture was roll-milled to form a viscous SMEDDS.

### EXAMPLE 56

According to the same method as in Experimental Example 4, a mixture containing 1 g of itraconazole, 2 g of citric acid and 4 g of Tween 80 was heat-melted or vacuum-melted and then cooled to 40° C. 4 g of Tween 80 and 3 g of lactic acid were added to the cooled mixture, and homogeneously mixed, and the resulting mixture was allowed to react at room temperature for about 24 hours. Thereafter, the mixture was roll-milled to form a viscous SMEDDS.

### EXAMPLE 57

According to the same method as in Experimental Example 4, a mixture containing 1 g of itraconazole, 2 g of citric acid and 4 g of Tween 80 was heat-melted or vacuum-melted and then cooled to 40° C. 6 g of Tween 80, 1 g of oleic acid and 2 g of effervescent sodium bicarbonate (NaHCO₃) were added to the cooled mixture, and homogeneously mixed, and the resulting mixture was allowed to react at room temperature for about 24 hours. Thereafter, the mixture was roll milled to form a viscous SMEDDS.

### EXAMPLE 58

The viscous semi-solid SMEDDS containing itraconazole prepared in Examples 1 to 57 was filled into soft gelatin capsules to prepare soft capsules containing itraconazole.

### EXAMPLE 59

The viscous semi-solid SMEDDS containing itraconazole prepared in Examples 1 to 57 was filled into hard gelatin capsules, and the connection parts of the capsules were sealed, thus giving hard capsules containing itraconazole.

### EXAMPLE 60

10 g of PVP was added to the mixture containing 10 g of the composition prepared in Example 50 and 10 ml of a mixture of acetone and ethanol at a volume ratio of 1:1. Thereafter the resulting mixture was dried in an oven at 70° C., to form a solid powdery preparation.

### EXAMPLE 61

10 g of the composition prepared in Example 50 was added to the 10 g of polyethylene glycol pre-heated at 60° C., and homogeneously mixed, and the resulting mixture was cooled at room temperature and dried to form a solid powdery preparation.

### EXAMPLE 62

10 g of the composition prepared in Example 50 and 10 g of PVP were mixed with 10 ml of a mixture of acetone and ethanol at a volume ratio of 1:1, and homogeneously dispered. After adding 5 ml of water thereto, the resulting mixture was spray-dried at 100° C. using a common spray-drier for pharmaceutical use, thereby forming a solid powdery preparation.

### EXAMPLE 63

10% of microcrystalline cellulose (Avicel) as a disintegrant and 2 % of colloidal silicon dioxide (Cab-O-Sil) as a lubricant were homogeneously mixed with solid powder prepared in Example 60-62. A weight amount corresponding to 50 mg of the drug was filled into an empty hard gelatin capsule, producing a solid capsule.

### EXAMPLE 64

10% of microcrystalline cellulose (Avicel) as a disintegrant and 2% of colloidal silicon dioxide (Cab-O-Sil) as a lubricant were homogeneously mixed with solid powder prepared in Example 60-62. A weight amount corresponding to 50 mg of the drug was tableted by a rotary tableting machine (12 stations, Korea Machine), producing a tablet.

### EXAMPLE 65

Tablets prepared in Example 64 were pulverized and then sieved using a 40-60 mesh to produce microgranules with a uniform size, in which micro powder was removed. A weight amount of microgranules corresponding to 50 mg of the drug was filled into an empty hydrogelatin capsule, producing solid capsules.

### COMPARATIVE EXAMPLE 1

According to the same method as in Experimental Example 1, a mixture containing 1 g of itraconazole, 2 g of citric acid and 2 g of Tween 80 was heat-melted or vacuum-melted. The resulting mixture was cooled and roll-milled to form a translucent viscous SMEDDS.

### COMPARATIVE EXAMPLE 2

A commercially available Sporanox capsule containing 100 mg of itraconazole was tested.

### COMPARATIVE EXAMPLE 3

A commercially available Sporanox capsule containing 100 mg of itraconazole was pulverized homogenously in the mortar to form a powder.

[Main Features of the Manufacturing Method for the SMEDDS According to the Present Invention]

The method of preparing SMEDDS according to the present invention is characterized in that a mixture comprising itraconazole and the surfactant contains organic acid or not, an additional step of roll-milling can be performed once or repeatedly, and the surfactant used at the step of heat-melting or vacuum-melting can be added twice after dividing it into two parts.

In case that no organic acid is contained in a mixture, the mixture comprising the drug, a surfactant and a fatty acid is heat-melted or vacuum-melted, and then roll-milled to produce a viscous SMEDDS as a final product (see, Experimental example 1). The drug and part of a surfactant were mixed at a weight ratio of 1:4-6 by heat-melting or vacuum-melting, and a first roll-milling step was carried out, and additionally the rest of the surfactant was added thereto, followed by a second roll-milling step, thus giving a viscous SMEDDS as a final product (see, Experimental example 2). On the other hand, in using a mixture containing organic acid, organic acid and a surfactant were mixed at a weight ratio of 1:2-4 relative to the used total amount of organic acid. If a final composition is in a viscous phase after cooling step, a roll-milling step is not performed (see, Experimental example 3). However, when being in a semi-solid wax phase, a final composition is converted into a viscous phase by roll-milling step, thus producing a viscous SMEDDS (see, Experimental example 4).

### EXPERIMENTAL EXAMPLE 1

### Preparation of SMEDDS Containing Itraconazole as Well as Fatty Acid and a Surfactant Through Melting and One-Step Roll Milling

A mixture comprising the drug, a surfactant and fatty acid was heat-melted or vacuum-melted at 150-160° C. for about 10 min. The resulting light brown viscous mixture was cooled to 40° C. According to intended use, a disintegrant (preferably, carmellose sodium), a co-solvent or cosurfactant (preferably, transcutol), and an anti-oxidant (preferably, butylated hydroxytoluene, added to an amount 0.1% of total weight of the mixture), were additionally added to the mixture, homogeneously dispersed by vortexing for about 10 min, and cooled at room temperature or −10° C. to give a semi-solid wax composition. After that, the wax composition was roll-milled once (one-step roll miling), thus forming a viscous semi-solid SMEDDS. No loss of the drug was detected during the overall process of preparing the final viscous semi-solid SMEDDS.

### EXPERIMENTAL EXAMPLE 2

### Preparation of SMEDDS Containing Itraconazole as Well as Fatty Acid and a Surfactant Through Melting and Two-Step Roll Milling

The drug and part of a surfactant were mixed at a weight ratio of 1:6, 1:7 or 1:8, together with fatty acid, and the mixture was heat-melted or vacuum-melted at 150-160° C. for about 10 min. The resulting yellow viscous mixture was cooled at room temperature or −10° C., and then a first roll-milling step was carried out to give a viscous semi-solid SMEDDS. The rest of the surfactant was added thereto, optionally with a disintegrant (preferably, carmellose sodium), a co-solvent or cosurfactant (preferably, transcutol), and an anti-oxidant (preferably, butylated hydroxytoluene, added to an amount 0.1% of the total weight of the mixture), and homogeneously dispersed by vortexing for about 10 min, and a second roll-milling step was carried out to form a viscous SMEDDS as a final composition. No loss of the drug was detected during the overall process of preparing the final viscous semi-solid SMEDDS.

### EXPERIMENTAL EXAMPLE 3

### Preparation of SMEDDS Containing Itraconazole as Well as Fatty Acid, a Surfactant and Organic Acid Through Melting and Mixing Without Roll Milling

Organic acid and part of a surfactant were mixed at a weight ratio of 1:2, and the mixture was heat-melted or vacuum-melted at 150-160° C. for about 10 min to form a yellow viscous mixture. The drug was then added thereto, followed by heat-melting or vacuum-melting at 150-160° C. for about 5 min and dissolving completely the drug to form a brown viscous mixture. After the resulting mixture was cooled to 40° C., the rest of the surfactant and fatty acid were added thereto, optionally with a disintergrnat (preferably, carmellose sodium), a co-solvent or cosurfactant (preferably, transcutol), and an anti-oxidant (preferably, butylated hydroxytoluene, added to an amount 0.1% of the total weight of the mixture), and homogeneously dispersed by vortexing for about 10 min, and cooled at room temperature or −10° C. to form a light brown viscous semi-solid SMEDDS. Loss of the drug was not found during the overall process of preparing the final viscous semi-solid SMEDDS.

### EXPERIMENTAL EXAMPLE 4

### Preparation of SMEDDS Containing Itraconazole as Well as Fatty Acid, a Surfactant and Organic Acid Through Melting and One-Step Roll Milling

Organic acid and part of a surfactant were mixed at a weight ratio of 1:2, and the mixture was heat-melted or melt-extruded at 150-160° C. for about 10 min to form a yellow viscous mixture. The drug was then added thereto, followed by heat-melting or vacuum-melting at 150-160° C. for about 5 min and dissolving completely the drug to form a brown viscous mixture. After the resulting mixture was cooled to 40° C., the rest of the surfactant and fatty acid were added thereto, optionally with a disintergrnat (preferably, carmellose sodium), a co-solvent or cosurfactant (preferably, transcutol), and an antioxidant (preferably, butylated hydroxytoluene, added to an amount 0.1% of the total weight of the mixture), and homogeneously dispersed by vortexing for about 10 min, and cooled at room temperature to form a light brown viscous mixture. The viscous mixture was left at room temperature for about 24 hours to transit to a viscous or semi-solid wax phase, followed by roll milling to form a viscous SMEDDS as a final composition. Loss of the drug was not found during the overall process of preparing the final viscous semi-solid SMEDDS.

### EXPERIMENTAL EXAMPLE 5

### Measurement of Content of Itraconazole Contained in the Viscous Semi-Solid Preparations

A pharmaceutical preparation containing itraconazole was completely dissolved in 500 ml of an ethanol solution containing phosphate-buffer (pH 6.8) in a volume amount of 50% (in case of containing slightly water-soluble materials, shaking incubation was performed for 10 min). The resulting mixture was centrifuged at 15,000 rpm for 2 min, and then filtered with a 0.45 μm membrane filter. After properly diluting 1 ml of the filtered solution, 20 μl of sample was used in quantification of itraconazole using HPLC, detected by monitoring UV absorbance at a wavelength of 263 nm, in which C18 ODS column (4.6×150 mm, 5 ∞m) was used, and a mixture of acetonitrile: 0.1% diethylamine (60:40 v/v%) was used for a mobile phase at a flow rate of 1 ml/min. The amount of injected sample was 20 μl. The HPLC consisted of a UV absorbance detector, a pump and an autosampler, connected to a computer with a Borwin program analyzing data. Concentration of itraconazole was determined using a standard curve based on peak area of cisapride used as an internal standard.

### EXPERIMENTAL EXAMPLE 6

### Changes in Physical Properties (Phase Separation, Color, Viscosity) of the Viscous Semi-Solid Preparations Containing Itraconazole During Storage at Room Temperature for Six Months

About 10 g of the viscous semi-solid itraconazole-containing preparations prepared in the above Examples was put into a test tube and stored at room temperature for six months. Changes in physical properties (phase separation, color, viscosity) were evaluated by naked eye. Table 1 is the result shown changes in physical properties (phase separation, color, viscosity) of the viscous semi-solid preparations containing itraconazole.

TABLE 1
Changes in phase separation, color and viscosity of the
viscous semi-solid preparations containing itraconazole
during storage

| | Phase | | Vis- | | Phase | | Vis- |
|---|---|---|---|---|---|---|---|
| Exp. | separation | Color | cosity | Example | separation | Color | cosity |
| 1 | + | + | + | 31 | 0 | ++ | ++ |
| 2 | + | + | + | 32 | 0 | ++ | ++ |
| 3 | + | + | + | 33 | 0 | ++ | ++ |
| 4 | + | + | + | 34 | 0 | ++ | ++ |
| 5 | + | ++ | + | 35 | 0 | ++ | ++ |
| 6 | + | + | + | 36 | 0 | ++ | ++ |
| 7 | + | + | + | 37 | 0 | ++ | ++ |
| 8 | + | + | + | 38 | 0 | ++ | ++ |
| 9 | + | ++ | ++ | 39 | 0 | ++ | ++ |
| 10 | + | ++ | ++ | 40 | 0 | + | ++ |
| 11 | + | ++ | ++ | 41 | 0 | + | ++ |
| 12 | ++ | + | +++ | 42 | 0 | + | ++ |
| 13 | +++ | + | +++ | 43 | 0 | + | ++ |
| 14 | +++ | + | +++ | 44 | 0 | + | ++ |
| 15 | +++ | + | +++ | 45 | 0 | + | ++ |
| 16 | +++ | + | +++ | 46 | + | + | + |
| 17 | +++ | + | +++ | 47 | + | + | + |
| 18 | +++ | + | ++ | 48 | 0 | + | + |
| 19 | + | + | + | 49 | + | + | + |
| 20 | + | + | + | 50 | 0 | + | + |
| 21 | + | + | + | 51 | + | ++ | + |
| 22 | + | + | + | 52 | + | +++ | + |
| 23 | + | + | + | 53 | 0 | ++ | + |
| 24 | + | + | + | 54 | 0 | ++ | + |
| 25 | + | + | + | 55 | 0 | +++ | + |
| 26 | + | + | + | 56 | 0 | +++ | + |
| 27 | + | + | + | 57 | + | ++ | + |
| 28 | + | + | + | | | | |
| 29 | 0 | ++ | ++ | | | | |
| 30 | 0 | ++ | ++ | | | | |

As shown in Table 1, it was found that physical properties (phase separation, color, viscosity) of the viscous semi-solid preparations containing itraconazole were largely influenced by their compositions (fatty acid and a surfactant, most preferably used or unused organic acid) and the amount of used component as well as the method of preparing the same (preferably whether roll-milling or not). Most preferably such a viscous composition comprising fatty acid, a surfactant and organic acid and being roll-milled showed excellent dissolution property, which will be described in detail in Experimental Example 7, below.

### EXPERIMENTAL EXAMPLE 7

### Measurement for Dissolution Rate of Itraconazole Contained in the Viscous Semi-Solid Preparations

Dissolution rate of itraconazole contained in the pharmaceutical preparation was analyzed according to the dissolution test method disclosed in a guidebook “Korea Pharmacopeia (7^{th} revision)”. A NaCl—HCl buffer solution (pH 1.4±0.1) was used as an artificial gastric juice, supplemented with Tween 80 in a volume ratio of 0.3% according to intended use. 0.02 M phosphate-buffered solution (pH 6.8±0.1) was used as an artificial intestinal juice. Dissolution was performed according to the paddle method at a stirring rate of 50 rpm and a dissolution temperature of 37±0.5° C., using 500 ml of dissolution solution. 0.5 ml samples were collected at 0, 2, 5, 10, 15, 30, 60, and 90 min, at which point the test solution was supplemented with an equivalent amount of dissolution solution. When being coagulated, the collected samples were applied to HPLC after being centrifuged at 15,000 rpm for 2 min and then filtered with a 0.45 μm membrane filter. 20 μl of sample was used in quantification of itraconazole using HPLC, detected by monitoring UV absorbance at 263 nm, in which C18 ODS column (4.6×150 mm, 5 m) was used, a mixture of acetonitrile: 0.1% diethylamine (60:40 v/v %) was used for a mobile phase at a flow rate of 1 ml/min. The HPLC consisted of a UV absorbance detector, a pump and an autosampler, connected to a computer with a Borwin program analyzing data. Concentration of itraconazole was determined using a standard curve based on peak area of cisapride used as an internal standard. The viscous semi-solid preparations prepared in the above four Examples were analyzed for dissolution rate (%) of itraconazole in an artificial gastric juice and artificial intestinal juice.

Tables 2 and 3, below, show dissolution rates (%) of itraconazole, which is contained in the viscous semi-solid preparations prepared using the composition comprising the drug, fatty acids and the surfactant according to the melting and one-step roll milling method in Experimental Example 1, in artificial gastric juice and artificial intestinal juice, respectively.

TABLE 2
Dissolution rates (%) of the viscous semi-solid
preparations containing 100 mg of itraconazole prepared
in Experimental Example 1 in artificial gastric juice

| | Time (hr) | | | | | |
|---|---|---|---|---|---|---|
| Exp. No. | 0.25 | 0.5 | 1 | 1.5 | 2 | 3 |
| Itraconazol | 0.753 | 1.879 | 1.965 | 1.974 | 1.948 | 2.044 |
| powder | | | | | | |
| Comparative | 2.757 | 3.361 | 3.580 | 4.121 | 4.138 | 4.994 |
| ex 1 | | | | | | |
| Comparative | 3.271 | 11.082 | 27.490 | 26.434 | 27.541 | 28.272 |
| ex 2 | | | | | | |
| (Sporanox ™ | | | | | | |
| capsule): | | | | | | |
| coagulation | | | | | | |
| was not | | | | | | |
| disentangled | | | | | | |
| Comparative | 37.229 | 57.635 | 60.968 | 60.180 | 59.609 | 56.711 |
| ex 2 | | | | | | |
| (Sporanox ™ | | | | | | |
| capsule): | | | | | | |
| coagulation | | | | | | |
| was | | | | | | |
| disintangled | | | | | | |
| during | | | | | | |
| dissolution | | | | | | |
| Ex 1 | 4.664 | 6.999 | 7.987 | 7.455 | 13.253 | 25.877 |
| Ex 2 | 4.661 | 5.744 | 7.234 | 8.401 | 10.685 | 13.269 |
| Ex 3 | 3.670 | 6.331 | 7.998 | 5.780 | 9.3528 | 11.651 |
| Ex 4 | 7.562 | 9.568 | 11.701 | 13.538 | 22.436 | 52.452 |
| Ex 5 | 4.053 | 5.112 | 5.473 | 5.493 | 6.821 | 7.638 |
| Ex 6 | 3.783 | 3.934 | 3.531 | 3.176 | 2.891 | 2.460 |
| Ex 7 | 2.766 | 3.152 | 3.179 | 2.970 | 2.851 | 2.583 |
| Ex 8 | 7.070 | 8.519 | 10.370 | 11.275 | 11.312 | 29.934 |
| Ex 19 | 2.747 | 2.791 | 4.203 | 6.499 | 8.489 | 17.260 |
| Ex 20 | 2.517 | 3.545 | 4.125 | 5.912 | 7.628 | 13.510 |
| Ex 21 | 3.305 | 2.961 | 4.068 | 5.333 | 6.946 | 11.616 |

TABLE 3
Dissolution rates (%) of the viscous semi-solid
preparations containing 100 mg of itraconazole prepared
in Experimental Example 1 in artificial interstinal juice

| | Time (hr) | | | | | |
|---|---|---|---|---|---|---|
| Exp. No. | 0.25 | 0.5 | 1 | 1.5 | 2 | 4 |
| Itraconazol | 0.655 | 0.819 | 0.708 | 1.582 | 1.496 | 1.521 |
| powder | | | | | | |
| Comparative | 5.149 | 7.175 | 7.062 | 8.846 | 10.456 | 19.615 |
| ex 1 | | | | | | |
| Comparative | 2.281 | 4.234 | 4.344 | 4.144 | 4.322 | 4.282 |
| ex 2 | | | | | | |
| (Sporanox ™ | | | | | | |
| capsule): | | | | | | |
| coagulation | | | | | | |
| was not | | | | | | |
| disentangled | | | | | | |
| Comparative | 2.281 | 1.468 | 2.011 | 1.754 | 1.122 | 1.084 |
| ex 2 | | | | | | |
| (Sporanox ™ | | | | | | |
| capsule): | | | | | | |
| coagulation | | | | | | |
| was | | | | | | |
| disintangled | | | | | | |
| during | | | | | | |
| dissolution | | | | | | |
| Ex 1 | 6.491 | 7.717 | 7.941 | 9.450 | 13.251 | 41.081 |
| Ex 2 | 5.693 | 7.950 | 90.64 | 10.144 | 12.564 | 21.930 |
| Ex 3 | 8.346 | 8.244 | 10.480 | 10.360 | 12.799 | 30.997 |
| Ex 4 | 7.562 | 9.568 | 11.701 | 13.538 | 22.436 | 72.665 |
| Ex 5 | 6.425 | 6.342 | 6.374 | 6.532 | 9.325 | 13.928 |
| Ex 6 | 1.654 | 3.927 | 3.801 | 5.035 | 3.969 | 3.630 |
| Ex 7 | 3.250 | 3.348 | 2.554 | 2.166 | 2.211 | 1.761 |
| Ex 8 | 10.109 | 12.170 | 14.815 | 16.107 | 20.446 | 64.347 |
| Ex 19 | 3.920 | 3.992 | 6.004 | 9.284 | 12.126 | 39.893 |
| Ex 20 | 3.596 | 3.939 | 5.863 | 8.449 | 10.898 | 41.454 |
| Ex 21 | 4.715 | 4.231 | 5.812 | 7.619 | 9.924 | 28.999 |

In case of the commercially available preparation (Sporanox™ capsule), coagulation of microgranules contained in the capsule occurred during the dissolution test. When the coagulation was removed, dissolution rate of itraconazole contained in Sporanox™ capsule was found to increase. This low dissolution rate of itraconazole contained in the commercially available preparation due to coagulation indicates that there is a potential for a large difference among individuals in the dissolution rate of itraconazole. Prepared in various Examples according to the method described in Experimental Example 1, the viscous semi-solid preparations showed dissolution rates similar to or lower than that of Sporanox™ capsule in artificial gastric juice. In contrast, the viscous semi-solid preparations showed dissolution rates higher than that of Sporanox™ capsule in artificial intestinal juice, where Sporanox™ capsule was found to have a very low dissolution rate.

Tables 4 and 5, below, show dissolution rates (%) of itraconazole, which is contained in the viscous semi-solid preparations prepared using the composition comprising the drug, fatty acids and the surfactant according to the melting and two-step roll milling method in Experimental Example 2, in artificial gastric juice and artificial intesinal juice, respectively.

TABLE 4
Dissolution rates (%) of the viscous semi-solid
preparations containing 100 mg of itraconazole prepared
in Experimental Example 2 in artificial gastric juice

| | Time(hr) | | | | | |
|---|---|---|---|---|---|---|
| Exp. No. | 0.25 | 0.5 | 1 | 1.5 | 2 | 4 |
| 22 | 3.010 | 2.861 | 5.437 | 11.085 | 16.735 | 32.833 |
| 23 | 4.768 | 10.323 | 20.261 | 26.621 | 33.274 | 45.152 |
| 24 | 7.789 | 11.315 | 20.650 | 27.679 | 33.905 | 41.007 |
| 25 | 6.362 | 15.267 | 22.334 | 29.191 | 37.353 | 50.787 |
| 26 | 3.190 | 5.180 | 10.712 | 16.811 | 24.017 | 44.723 |
| 27 | 3.205 | 7.326 | 15.768 | 24.151 | 32.960 | 50.616 |
| 28 | 5.4027 | 10.065 | 19.228 | 28.543 | 36.780 | 50.244 |

TABLE 5
Dissolution rates (%) of the viscous semi-solid
preparations containing 100 mg of itraconazole prepared
in Experimental Example 2 in artificial intestinal juice

| | Time(hr) | | | | | |
|---|---|---|---|---|---|---|
| Exp. No. | 0.25 | 0.5 | 1 | 1.5 | 2 | 4 |
| 22 | 4.301 | 4.092 | 7.768 | 15.836 | 23.908 | 74.164 |
| 23 | 5.231 | 13.915 | 31.575 | 49.381 | 61.800 | 98.219 |
| 24 | 15.578 | 22.630 | 41.300 | 55.358 | 67.810 | 90.681 |
| 25 | 4.762 | 12.667 | 35.127 | 43.195 | 63.253 | 96.877 |
| 26 | 4.568 | 7.430 | 15.361 | 23.982 | 34.310 | 82.041 |
| 27 | 6.441 | 12.270 | 26.328 | 40.252 | 54.936 | 84.360 |
| 28 | 9.004 | 16.777 | 32.095 | 47.572 | 61.390 | 86.825 |

Prepared in various Examples by heat-melting or vacuum-melting, then two-step roll milling, and then cooling, according to the method described in Experimental Example 2, the viscous semi-solid preparations were found to be remarkably increased in dissolution rate in artificial intestinal juice in comparison with Sporanox™ capsule, depending on the use of fatty acids and the surfactant and concentration thereof, indicating that the two-step roll milling process capable of supplying a homogeneously dispersed mixture positively affects dissolution rate of itraconazole. In addition, when containing the disintegrant or the foaming agent, the viscous semi-solid preparations were found to have high initial dissolution rates.

Collectively, when being prepared by roll milling a composition comprising a surfactant and fatty acids, viscous semi-solid preparations have excellent dissolution rates.

Tables 6 and 7, below, show dissolution rates (%) of itraconazole, which is contained in the viscous semi-solid preparations prepared using the composition comprising the drug, fatty acids and the surfactant as well as organic acids according to the melting and mixing method in Experimental Example 3, in artificial gastric juice and artificial intestinal juice, respectively.

TABLE 6
Dissolution rates (%) of the viscous semi-solid
preparations containing itraconazole prepared in
Experimental Example 3 in artificial gastric juice

| | Time(hr) | | | | | |
|---|---|---|---|---|---|---|
| Exp. No. | 0.25 | 0.5 | 1 | 1.5 | 2 | 4 |
| 29 | 30.225 | 33.667 | 50.245 | 67.334 | 79.178 | 81.845 |
| 30 | 21.554 | 27.124 | 42.985 | 46.465 | 50.385 | 58.116 |
| 31 | 26.257 | 30.663 | 46.768 | 60.256 | 77.229 | 75.459 |
| 32 | 22.297 | 25.367 | 33.568 | 39.889 | 44.690 | 46.961 |
| 33 | 33.579 | 40.129 | 57.079 | 70.448 | 77.815 | 78.529 |
| 34 | 37.669 | 45.123 | 54.203 | 68.554 | 70.699 | 71.869 |
| 35 | 35.221 | 54.556 | 83.287 | 85.443 | 89.864 | 93.261 |
| 36 | 26.112 | 40.779 | 69.257 | 75.108 | 76.538 | 79.438 |
| 37 | 20.011 | 23.871 | 26.354 | 46.078 | 49.174 | 50.014 |
| 38 | 32.114 | 51.657 | 60.134 | 65.321 | 66.727 | 78.267 |
| 39 | 40.126 | 51.002 | 60.591 | 64.554 | 66.120 | 68.121 |
| 40 | 41.551 | 56.885 | 63.358 | 63.584 | 65.970 | 67.661 |

TABLE 7
Dissolution rates (%) of the viscous semi-solid
preparations containing itraconazole prepared in
Experimental Example 3 in artificial intestinal juice

| | Time(hr) | | | | | |
|---|---|---|---|---|---|---|
| Exp. No. | 0.25 | 0.5 | 1 | 1.5 | 2 | 4 |
| 29 | 40.080 | 53.214 | 68.569 | 70.212 | 76.044 | 77.896 |
| 30 | 39.021 | 50.321 | 65.654 | 70.998 | 72.963 | 78.036 |
| 31 | 45.587 | 66.667 | 76.509 | 78.181 | 70.669 | 40.391 |
| 32 | 35.621 | 40.222 | 50.884 | 58.121 | 65.509 | 68.611 |
| 33 | 40.691 | 57.441 | 79.678 | 81.514 | 70.554 | 38.078 |
| 34 | 50.655 | 67.112 | 75.468 | 81.372 | 54.222 | 19.227 |
| 35 | 43.212 | 85.296 | 78.093 | 50.664 | 26.075 | 23.121 |
| 36 | 52.323 | 74.949 | 63.967 | 40.154 | 26.549 | 22.034 |
| 37 | 33.914 | 20.272 | 19.445 | 18.212 | 15.555 | 10.011 |
| 38 | 55.333 | 77.907 | 94.242 | 90.396 | 80.279 | 38.446 |
| 39 | 33.661 | 56.441 | 59.308 | 68.397 | 75.212 | 78.665 |
| 40 | 30.557 | 47.240 | 59.010 | 72.894 | 75.664 | 80.441 |

Despite not being roll-milled, the viscous semi-solid preparations prepared according to the method described in Experimental Example 3 showed initial dissolution rate superior to the commercially available preparation and the Comparative Examples, with a similar dissolution pattern. Especially in the preparations of Examples 39 and 40 using the simple process without the roll-milling step, they were found to form a clear viscous phase having good physicochemical properties, and be relatively stable at high temperatures, as well as having stable physicochemical properties.

Also, dissolution rates (%) of itraconazole, which is contained in the viscous semi-solid preparations prepared according to the method described in Experimental Example 4, were evaluated in artificial gastric juice and artificial intestinal juice, and the results are given in Tables 8 and 9, below, respectively.

TABLE 8
Dissolution rates (%) of the viscous semi-solid
preparations containing itraconazole prepared in
Experimental Example 4 in artificial gastric juice

| | Time(hr) | | | | | |
|---|---|---|---|---|---|---|
| Exp. No. | 0.25 | 0.5 | 1 | 1.5 | 2 | 4 |
| 29 | 40.080 | 53.214 | 68.569 | 70.212 | 76.044 | 77.896 |
| 30 | 39.021 | 50.321 | 65.654 | 70.998 | 72.963 | 78.036 |
| 31 | 45.587 | 66.667 | 76.509 | 78.181 | 70.669 | 40.391 |
| 32 | 35.621 | 40.222 | 50.884 | 58.121 | 65.509 | 68.611 |
| 33 | 40.691 | 57.441 | 79.678 | 81.514 | 70.554 | 38.078 |
| 34 | 50.655 | 67.112 | 75.468 | 81.372 | 54.222 | 19.227 |
| 35 | 43.212 | 85.296 | 78.093 | 50.664 | 26.075 | 23.121 |
| 36 | 52.323 | 74.949 | 63.967 | 40.154 | 26.549 | 22.034 |
| 37 | 33.914 | 20.272 | 19.445 | 18.212 | 15.555 | 10.011 |
| 38 | 55.333 | 77.907 | 94.242 | 90.396 | 80.279 | 38.446 |
| 39 | 33.661 | 56.441 | 59.308 | 68.397 | 75.212 | 78.665 |
| 40 | 30.557 | 47.240 | 59.010 | 72.894 | 75.664 | 80.441 |

TABLE 9
Dissolution rates (%) of the viscous semi-solid
preparations containing itraconazole prepared in
Experimental Example 4 in artificial intestinal juice

| | Time(hr) | | | | | |
|---|---|---|---|---|---|---|
| Exp. No. | 0.25 | 0.5 | 1 | 1.5 | 2 | 4 |
| 50 | 64.036 | 74.392 | 79.276 | 79.978 | 81.826 | 93.677 |
| 48 | 59.142 | 67.697 | 69.714 | 74.173 | 77.531 | 95.911 |
| 49 | 60.328 | 67.706 | 72.829 | 77.104 | 90.556 | 98.699 |
| 47 | 62.333 | 70.113 | 75.065 | 81.111 | 92.398 | 98.777 |
| 52 | 66.884 | 75.222 | 84.241 | 86.121 | 90.444 | 95.621 |
| 53 | 71.870 | 74.400 | 77.667 | 94.573 | 97.264 | 98.555 |
| 54 | 67.804 | 78.452 | 87.986 | 95.468 | 98.425 | 98.821 |
| 55 | 52.088 | 64.416 | 70.969 | 75.922 | 81.209 | 97.644 |
| 56 | 49.537 | 60.637 | 68.587 | 70.950 | 79.458 | 98.323 |

When containing organic acid, the viscous SMEDDS was found to be more stable and have increased dissolution rate. In detail, the viscous SMEDDS prepared in Examples 50 to 56, which contain fatty acid, the surfactant and organic acid, and which were roll-milled, showed good dissolution rates. Especially, the viscous semi-solid preparations prepared according to the method in Experimental Example 4 was found to have stable physical and chemical properties during storage for a short or long period of time, with no appearance of precipitates and no change in phase separation and color, in addition to having excellent dissolution rates in artificial gastric juice and especially in artificial intestinal juice.

### EXPERIMENTAL EXAMPLE 8

### Comparison of Plasma Concentrations of Itraconazole Contained in the Viscous Semi-Solid Preparations of the Present Invention and the Commercially Available Preparation in Mice

After male white mice (Sprague-Dawley) having body weights of 250-310 g, purchased from Korea National Institute of Health, were adjusted to a new environment for about 1-2 weeks, mice were fasted for one day before the experiment and anesthetized with ether, and cannulation to the left femoral artery was performed using a tube connected to a syringe containing 50 IU/ml heparin. When the mice come out from the anesthesia after about 2 hours, a suspension of the solid powdery preparation according to the present invention or the commercially available preparation was administered to mice using a sonde for oral administraton in an amount of 20 mg itraconazole per kg, and blood was then collected from left femur artery at 0.25, 0.5, 1, 2, 3, 4, 5, 6 and 8 hrs after administraton. The collected blood was centrifuged at 3500 rpm for 10 min, and the isolated blood plasma was stored at −20° C. until analysis. To determine concentration of itraconazole in blood, HPLC was carried out as follows. 300 μl of blood was put into a microtube, and 50 μl of a solution containing an internal standard substance and 600 μl of acetonitrile were then added to the tube, followed by vortexing for 2 min and then centrifuging at 15,000 rpm for 2 min. 20 μl of the supernatant was applied to HPLC, while monitoring UV absorbance at 263 nm, in which C18 ODS column (4.6×150 mm, 5 m) was used, a mixture of acetonitrile: 0.1% diethylamine (60:40 v/v%) was used for a mobile phase at a flow rate of 1 ml/min. The HPLC consisted of a UV absorbance detector, a pump and an autosampler connected to a computer with a Borwin program analyzing data. Concentration of itraconazole was determined using a standard curve based on peak area of cisapride used as an internal standard.

After orally administering to rats the viscous soft capsules according to the present invention and the commercially available preparation (Sporanox™ capsule), which are containing itraconazole, plasma concentrations of itraconazole were investigated with the lapse of time, and the results are given in Table 10, below.

TABLE 10
Comparison of plasma concentration (μg/ml) of
itraconazole according to time after oral administration
of the viscous soft capsules according to the present
invention and Sporanox ™ capsule containing itraconazole

| | Time(hr) | | | | | | | | |
|---|---|---|---|---|---|---|---|---|---|
| Exp. No. | 0.25 | 0.5 | 1 | 2 | 3 | 4 | 5 | 6 | 8 |
| C. 3 | 0.098 | 0.191 | 0.213 | 0.310 | 0.320 | 0.282 | 0.273 | 0.262 | 0.197 |
| (Sporanox ™) | | | | | | | | | |
| 14 | 0.091 | 0.264 | 0.382 | 0.398 | 0.369 | 0.279 | 0.245 | 0.242 | 0.210 |
| 23 | 0.171 | 0.343 | 0.663 | 0.689 | 0.673 | 0.535 | 0.469 | 0.393 | 0.354 |
| 25 | 0.349 | 0.451 | 0.618 | 0.814 | 0.928 | 0.553 | 0.493 | 0.472 | 0.382 |
| 39 | 0.305 | 0.619 | 0.858 | 0.817 | 0.605 | 0.612 | 0.506 | 0.463 | 0.407 |
| 40 | 0.382 | 0.795 | 0.925 | 1.043 | 0.826 | 0.763 | 0.733 | 0.644 | 0.582 |
| 50 | 0.350 | 0.471 | 0.786 | 0.719 | 0.709 | 0.665 | 0.559 | 0.483 | 0.444 |

As shown in Table 10, after oral administration of the viscous soft capsules prepared in all Examples except Example 14, plasma concentrations of itracozole were higher than that of the commercially available capsule. Especially, when orally administering to rats the viscous soft capsules prepared in Examples 39, 40 and 50, maximum plasma concentrations (Cₘₐₓ) of itraconazole and area-under-the-curve (AUC) were observed to be very high, indicating the viscous SMEDDS according to the present invention improve bioavailability of itracozole.

### EXPERIMENTAL EXAMPLE 9

### Comparison of Plasma Concentration of Itraconazole Contained in the Viscous Semi-Solid Preparations of the Present Invention and the Commercially Available Preparation in Human

Based on the criterion of the biological equivalence test (2×2 cross-over, 3 weeks washout period), the capsule containing 40 mg of itraconazole according to the present invention and Sporanox™ capsule containing itraconazole were orally administered to 6 healthy fasted human adult males aged 20-40, together with 300 ml water. 0, 1, 2, 3, 4, 5, 6, 8, 12 and 24 hrs after the administration, 10 ml blood was collected from their arms using catheters, and put into vacutainer tubes, followed by addition of heparin to prevent clotting. The volunteers were allowed to take small drinks after 3 hr and Gimbap, rice roll with seaweed that is a kind of Korean food, after 10 hr. 8 hr and 10 hr after the administration, drinks and a bowl of boiled rice mixed with some vegetables respectively were supplied to the subjects. During the experiment, drinking of alcoholic beverages or caffeine was prohibited, and activity was limited to reading and sleeping. The collected blood was centrifuged at 3500 rpm for 10 min, and the isolated blood plasma using iron-free tubes was stored at −20° C. until analysis. To determine concentration of itraconazole in blood, HPLC was carried out as follows. 300 μl of blood was put into a microtube, and 50 μl of a solution containing an internal standard substance and 600 μl of acetonitrile (CH₃CN) were then added to the tube, followed by vortexing for 2 min and then centrifuging at 15,000 rpm for 2 min. 20 μl of the supernatant was applied to HPLC, while monitoring UV absorbance at 263 nm, in which C18 ODS column (4.6×150 mm, 5 μm) was used, a mixture of acetonitryl: 0.1% diethylamine (60:40 v/v %) was used for a mobile phase, and a flow rate was 1 ml/min. The used HPLC consists of a UV absorbance detector, a pump and an autosampler, and connected to a computer with a Borwin program analyzing data. Concentration of itraconazole was determined using a standard curve based on peak area of cisapride used as an internal standard.

After orally administering to humans the viscous soft capsules prepared according to the method in Experimental Example 2, which contains 60 mg of itraconazole, and the commercially available preparation (Sporanox™ capsule), which contains 100 mg of itraconazole, plasma concentrations of itraconazole was investigated with the elapse of time, and the results are given in Table 11, below.

TABLE 11
Comparison of plasma concentrations (μg/ml) of
itraconazole in humans according to time after oral
administration of the viscous soft capsules containing 60 mg
of itraconazole and Sporanox ™ capsule containing 100 mg
of itraconazole

| | Time(hr) | | | | |
|---|---|---|---|---|---|
| Exp. No. | 0 | 1 | 2 | 3 | 4 |
| 25 | 0.0094 | 0.0441 | 0.0340 | 0.0275 | 0.0217 |
| C. 2 | 0.0035 | 0.0173 | 0.0163 | 0.0175 | 0.0159 |
| (Sporanox ™) | | | | | |
| | Time(hr) | | | | |
| Exp. No. | 5 | 6 | 8 | 12 | 24 |
| 25 | 0.0138 | 0.0088 | 0.0052 | 0.0015 | 0.0000 |
| C. 2 | 0.0111 | 0.0067 | 0.0040 | 0.0016 | 0.0000 |
| (Sporanox ™) | | | | | |

When the capsule filled with the composition prepared in Example 25 was orally administered to human beings at dosages lower than that of Sporanox™ capsule, which showed a high dissolution rate as seen in Tables 2 and 3, it showed Cₘₐₓ and AUC values higher than Sporanox™ capsule, indicating improved bioavailability of itraconazole.

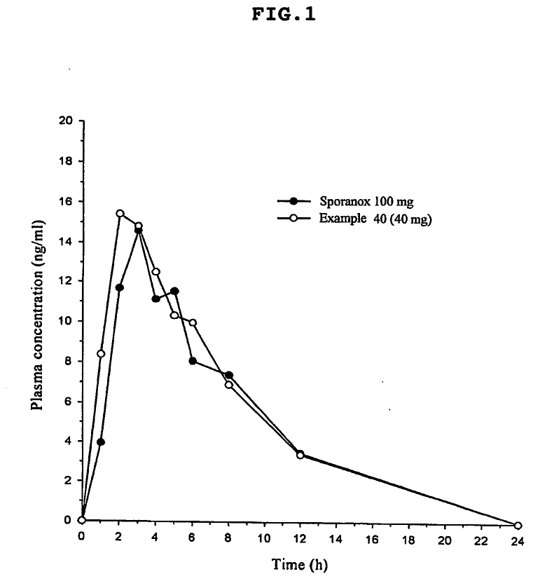

In addition, bioavailability of capsules containing the stable composition prepared in Example 40 according to the simple process in Experimental Example 3 was evaluated in comparison with that of the commercially available capsule. After orally administering to human the viscous soft capsules prepared according to the method in Experimental Example 2, which contains 40 mg of itraconazole, and the commercially available preparation (Sporanox™ capsule), which are containing 100 mg of itraconazole, plasma concentrations of itraconazole were investigated with the lapse of time, and the results are given in Table 12, below, and FIG. 1. Pharmacokinetic parameters were obtained from the data in Table 12, and the results are given in Table 13, below.

TABLE 12
Comparison of plasma concentrations (μg/ml) of
itraconazole in humans according to time after oral
administration of the viscous soft capsules containing 40 mg
of itraconazole and Sporanox ™ capsule containing 100 mg
of itraconazole

| | Time(hr) | | | | |
|---|---|---|---|---|---|
| Exp. No. | 1 | 2 | 3 | 4 | 5 |
| C. 2 | 0.00395 | 0.01168 | 0.01460 | 0.01113 | 0.01153 |
| (Sporanox ™) | | | | | |
| 40 | 0.00838 | 0.01542 | 0.01482 | 0.01303 | 0.01203 |

Time(hr)

| Exp. No. | 6 | 8 | 12 | 24 |
|---|---|---|---|---|
| C. 2 | 0.00808 | 0.00740 | 0.00348 | 0.0000 |
| (Sporanox ™) | | | | |
| 40 | 0.01067 | 0.00692 | 0.00340 | 0.0000 |

TABLE 13
Comparison of pharmacokinetic parameters

| | | | AUC |
|---|---|---|---|
| | Cₘₐₓ (μg/ml) | Tₘₐₓ (hr) | (μg · hr/ml) |
| C.3 (Sporanox ™) | 0.015 ± 0.011 | 3.33 ± 0.9 | 0.115 ± 0.10 |
| 40 | 0.016 ± 0.02 | 2.33 ± 0.12 | 0.124 ± 0.05 |

As shown in Table 13, the capsule preparation containing 40 mg of itraconazole and the commercially available capsule containing 100 mg of itraconazole differed by only ±20% in their pharmacokinetics. As shown in FIG. 1, the two capsule preparations showed very similar plasma concentrations of itraconazole, displaying a biologically equivalent behavior.

The capsule preparation prepared in Example 50 according to the procedure in Experimental Example 4 showed good stability and dissolution rate was found to have plasma concentrations of itraconazole very similar to that of the capsule prepared in Example 40. In addition, the viscous SMEDDS containing fatty acids, the surfactant and an organic acid, which is prepared by roll-milling in Example 50, was found to have excellent bioavailability, as will be described in detail in Experimental Example 10, below.

### EXPERIMENTAL EXAMPLE 10

### Test for Stability of Itraconazole Contained in Capsules

The viscous semi-solid preparations prepared in many Examples selected based on physicochemical properties (phase transition, dissolution rate, etc.) were tested for stability for a short or long period of time. The viscous semi-solid capsules containing itraconazole, prepared in the Examples were put into a plastic bottle along with a drying agent, and then covered with a cap, without other auxiliary apparatuses. The bottle was left at 40° C. under 75% humidity. To estimate stability of itraconazole at the starting point and after 6 months, content of itraconazole in the capsule, phase transition and dissolution rate were investigated according to the same method as in Experimental examples 5, 6 and 7, respectively. Separately, some viscous semi-solid preparations not being filled into capsules were placed onto petri dishes without being covered, and the petri dishes were placed at 40° C. under 75% humidity, and content of itraconazole in the capsule, phase transition and dissolution rates were determined.

Additionally, after the viscous semi-solid preparation prepared in Example 50 was stored at 40° C. under 75% humidity for six months, stability of itraconazole in artificial gastric juice was evaluated by measuring the size of particles using a particle counter (Par III, Ozuka, Japan) based on the laser dynamic scattering principle.

When the stability test was performed at 40° C. under 75% humidity after storage for six months after putting the viscous semi-solid preparations into a plastic bottle or exposing them on petric dishes, the content of itraconazole was almost the same in all viscous semi-solid preparations prepared in Examples. This result indicates that itraconazole is very stable at high temperatures.

After storage for six months, change in dissolution rate of itraconazole contained in the viscous soft capsules was investigated, and the results are given in Table 14, below.

TABLE 14
Change in dissolution rate after storage of the viscous
soft capsules containing itraconazole at 40° C. under 75%
humidity for six months

| | | Dis- | Artificial | | Artificial | |
|---|---|---|---|---|---|---|
| | | solution | gastric juice | | intestinal juice | |
| | Storage | Storage | Immediately | | Immediately | |
| Exp. | vessel/ | Time | after | After 6 | after | After 6 |
| No. | period | (hr) | prep. | months | prep. | months |
| 23 | Capsule | 0.5 | 10.3 | 2.0 | 13.9 | 5.3 |
| | (6 | 1 | 20.3 | 7.2 | 31.6 | 8.8 |
| | months) | 1.5 | 26.6 | 13.3 | 49.4 | 74.9 |
| | | 2 | 33.2 | 15.9 | 61.8 | N/A |
| 25 | Capsule | 0.5 | 15.3 | 10.9 | 12.7 | 6.2 |
| | (6 | 1 | 22.3 | 14.4 | 35.1 | 8.6 |
| | months) | 1.5 | 29.1 | 13.2 | 43.2 | 8.5 |
| | | 2 | 37.4 | 16.7 | 63.3 | 7.0 |
| 35 | Petri | 0.5 | 54.6 | N/A | 85.3 | 72.3 |
| | dish | 1 | 83.3 | 89.4 | 78.1 | 55.6 |
| | (2 | 1.5 | 85.4 | 91.4 | 50.7 | N/A |
| | weeks) | 2 | 89.9 | 94.5 | 26.1 | 8.3 |
| 36 | Petri | 0.5 | 40.8 | N/A | 74.9 | 69.9 |
| | dish | 1 | 69.3 | 69.2 | 63.9 | 63.8 |
| | (2 | 1.5 | 75.1 | 75.1 | 40.2 | N/A |
| | weeks) | 2 | 76.5 | 79.4 | 26.5 | 13.5 |
| 38 | Petri | 0.5 | 51.7 | N/A | 77.9 | 74.2 |
| | dish | 1 | 60.1 | 70.1 | 94.2 | 81.7 |
| | (2 | 1.5 | 65.3 | 78.8 | 90.4 | N/A |
| | weeks) | 2 | 66.7 | 83.4 | 80.3 | 38.4 |
| 40 | Capsule | 0.5 | 56.9 | 25.8 | 47.2 | 56.5 |
| | (6 | 1 | 63.4 | 27.9 | 59.0 | 64.2 |
| | months) | 1.5 | 63.6 | 29.8 | 72.9 | 66.7 |
| | | 2 | 66.0 | 35.3 | 75.7 | 67.2 |
| 50 | Capsule | 0.5 | 85.5 | 69.6 | 74.4 | 58.5 |
| | (6 | 1 | 93.2 | 76.9 | 79.3 | 69.0 |
| | months) | 1.5 | 95.0 | 87.3 | 80.0 | 76.9 |
| | | 2 | 96.8 | 93.0 | 81.8 | 79.4 |
| 54 | Petri | 0.5 | 84.8 | 57.6 | 78.5 | 28.0 |
| | dish | 1 | 86.9 | 71.1 | 88.0 | 32.7 |
| | (2 | 1.5 | 90.7 | 75.3 | 95.5 | 33.3 |
| | weeks) | 2 | 90.7 | 76.9 | 98.4 | 38.1 |
| 56 | Petri | 0.5 | 66.2 | 34.0 | 60.6 | 31.3 |
| | dish | 1 | 69.6 | 39.3 | 68.6 | 32.1 |
| | (2 | 1.5 | 70.2 | 40.6 | 71.0 | 37.5 |
| | weeks) | 2 | 67.7 | 51.4 | 79.5 | 38.4 |

Note:
N/A: Not Available

When the viscous semi-solid preparations prepared in Examples 35, 36 and 38 were orally administered, it was found that they were generally stable in artificial gastric juice. However, dissolution rate was remarkably reduced in artificial intestinal juice compared to that at the early stage, in which recrystallization was detected, indicating that the preparations are physically unstable.

When the viscous semi-solid preparations prepared in Examples 39 and 40 through the simple process were orally administered, initial dissolution rate was observed to be slightly reduced in artificial gastric juice. Dissolution patterns of the said preparations was stable. However, they showed dissolution rates higher than that of the commercially available capsule, and dissolution pattern similar to the commercially available capsule. In addition, they were relatively stable at high temperature, not changed in physical properties including precipitation of the drug, phase separation and color, and very stable chemically. However, when being stored for a long period of time, they were found to release the drug slowly, thus delaying dissolution of the drug.

When the viscous semi-solid preparation prepared in Experimental example 4 was orally administered, a high initial dissolution rate was observed in artificial gastric juice and intestinal juice, which gradually reduced after storage for a long period of time, but physical properties were not influenced. In contrast, the viscous semi-solid preparation prepared in Example 50 was found to be highly stable with a dissolution rate relatively higher than that of other semi-solid preparations, as well as stable physical properties.

On the other hand, when being dispersed in artificial gastric juice, the SMEDDS prepared in Example 50 was found to be very stable, although size of particles was increased from 214 to 395 μm after storage for six months at room temperature. When the SMEDDS was dispersed in water, it was observed that microemulsion was formed.

As apparent in the data for stability and dissolution rate of the viscous semi-solid preparations prepared in several Examples, the viscous semi-solid preparations containing poorly water-soluble itraconazole have improved stability and bioavailability, thus being able to replace the commercially available preparation.

### Industrial Applicability

As described hereinbefore, the composition according to the present invention is economical thanks to the simple process of simply heat-melting or vacuum-melting the components contained in the composition with no use of organic solvents, as well as being very stable at high temperatures because of not containing unstable components such as sugars including sucrose, glucose, lactose, mannitol, sorbitol and fructose. In particular, as apparent from the data of dissolution rate in artificial gastric juice and intestinal juice, the composition of the present invention has a remarkably high dissolution rate in comparison with the commercially available preparation (Sporanox™ capsule). As apparent from the data of plasma concentrations of itraconazole, evaluated after oral administration, the composition of the present invention has 2 to 6 times higher bioavailability than the commercially available preparation (Sporanox™ capsule), while giving plasma concentrations of itraconazole similar to that of the commercially available capsule. In detail, when the composition of the present invention is administered at a dosage of 30-80 mg once daily, its efficacy is equivalent to that of 100 mg of the commercially available preparation, thus allowing replacement of the commercially available preparation with the composition of the present invention. Further, a composition with high bioavailability according to the present invention can be formulated as a solid powder by mixing with a base capable of being formulated into soft capsules, hard capsules and solid powder, and then drying the mixture, which may be filled into capsules by adding pharmaceutically acceptable additives to the solid powder preparation, or other orally administrable preparations such as compressed particles, pellets, or tablets.

What is claimed is:

1. A composition in a viscous phase, comprising poorly water-soluble itraconazole in an amount of 8-12 parts by weight, fatty acid or fatty alcohol in an amount of 8-60 parts by weight and a surfactant in an amount of 64-120 parts by weight, forming self-microemulsifying drug delivery system(SMEDDS) when administered to a human body:

2. (deleted)

3. The composition according to claim 1, wherein the fatty acid or fatty alcohol is selected from the group consisting of oleic acid, stearyl alcohol, myristic acid, linoleic acid or lauric acid, capric acid, caprylic acid, caproic acid, and mixtures thereof.

4. (deleted)

5. (deleted)

6. The composition according to claim 1, wherein the surfactant is selected from the group consisting of sodium lauryl sulfate and its derivatives, poloxamer and its derivatives, labrafil, labrafac, polysorbate, sorbitan esters, cremophor, PEG-60 hydrogenated castor oil, PEG-40 hydrogenated castor oil, sodium lauryl glutamate, disodium cocoamphodiacetate, and mixtures thereof.

7. (deleted)

8. (deleted)

9. The composition according to claim 6, wherein the surfactant is selected from -the group consisting of Tween 20, Tween 80, and mixtures thereof.

10. The composition according to claim 1, further comprising a cosurfactant, wherein the cosurfactant is selected from the group consisting of polyethylene glycol and its derivatives, ethanol-containing alcohols, transcutol, propylene glycol, ethyl oleate, methyl pyrrolidone, ethyl pyrrolidone, propyl pyrrolidinone, glycerol, xylitol, sorbitol, dextrose, mannitol, and mixtures thereof.

11. (deleted)

12. (deleted)

13. The composition according to claim 1, further comprising one or more organic acids in an amount of 16-24 parts by weight.

14. The composition according to claim 13, wherein the organic acids are selected from the group consisting of citric acid, fumaric acid, maleic acid, malic acid, salicylic acid, formic acid, glycolic acid, lactic acid, acetic acid, propionic acid, α- and β-hydroxy acid, mixtures thereof.

15. The composition according to claim 13, wherein the organic acid is citric acid.

16. The composition according to claim 1, further comprising one or more selected from the group consisting of oil, an anti-oxidant, a disintegrant and a foaming agent.

17. The composition according to claim 16, wherein the oil is selected from the group consisting of caprylic/capric triglyceride, α-bisabolol, tocopherol acetate, liposome, phospholipid including phosphatidylcholine, di-C₁₂₋₁₃ alkyl malate, coco-caprylate/caprate, cetyl octanoate, and hydrogenated castor oil.

18. The composition according to claim 16, wherein the anti-oxidant is selected from the group consisting of butylated hydroxytoluene (BHT), sodium bisulfite, α-tocopherol, vitamin C, β-carotene, ascobylpamitate, tocopherol acetate, fumaric acid, nalic acid, butylated hydroxyanisole, propyl gallate, and sodium ascorbate.

19. (deleted)

20. (deleted)

21. (deleted)

22. The composition according to claim 13, comprising itraconazole in an amount of 8-12 parts by weight, oleic acid in an amount of 8-60 parts by weight, Tween 20 or 80 parts in an amount of 64-120 parts by weight, and citric acid in an amount of 16-24 parts by weight.

23. The composition according to claim 13, comprising itraconazole in an amount of 8-12 parts by weight, fatty acid selected from the group consisting of oleic acid, lauric acid, caprylic acid and mixtures thereof in an amount of 40-60 parts by weight, Tween 20 or 80 in an amount of 64-96 parts by weight, and citric acid in an amount of 16-24 parts by weight.

24. The composition according to claim 23, wherein the fatty acid is a mixture of lauric acid and caprylic acid, and contained in an amount of 40-60 parts by weight.

25. The composition according to claim 23, wherein the fatty acid is a mixture of lauric acid and caprylic acid, and lauric acid is contained in an amount of 8-12 parts by weight and caprylic acid in an amount of 32-48 parts by weight.

26. A soft capsule preparation filled with a composition according to any one of claims 1 to 25.

27. The soft capsule preparation according to claim 26, comprising 30-120 mg of itraconazole.

28. (deleted)

29. (deleted)

30. A hard capsule preparation filled with a composition according to any one of claims 1 to 25.

31. The hard capsule preparation according to claim 30, comprising 30-120 mg of itraconazole.

32. (deleted)

33. (deleted)

34. A pharmaceutical preparation formulated into a solid powder, which is prepared by mixing a composition according to any one of claims 1 to 25 with a base, and melting and drying the mixture to pulverize it, or into compressed granules, pellets or capsules, which are prepared by additionally compressing or formulating said solid powder.

35. The pharmaceutical preparation according to claim 34, wherein the base is a polymeric base.

36. The pharmaceutical preparation according to claim 35, wherein the polymeric base is selected from the group consisting of polyethylene glycol, carbowax, and polyvinyl pyrrolidone.

37. The pharmaceutical preparation according to claim 35, wherein the base further comprises a water-soluble base.

38. The pharmaceutical preparation according to claim 37, wherein the water-soluble base is selected from the group consisting of gelatin, gums, carbohydrates, cellulose and its derivatives, polyethylene oxide and its derivatives, polyvinyl alcohol, polyacrylic acid and its derivatives, polymethylacrylate, and inorganic compounds.

39. The pharmaceutical preparation according to claim 34, comprising 30-120 mg of itraconazole.

40. (deleted)

41. (deleted)

42. A method of preparing a composition according to claim 1, comprising the steps of:
heat-melting or vacuum-melting a mixture of itraconazole, fatty acid or fatty alcohol and a surfactant; and
cooling the melted mixture.

43. The method according to claim 42, further comprising a step of milling after cooling.

44. The method according to claim 42, wherein the mixture further comprises organic acid.

45. (deleted)

46. A method of preparing a composition according to claim 13, comprising the steps of:
heat-melting or vacuum-melting a mixture of itraconazole, organic acid and a surfactant;
cooling the melted mixture to 40° C.;
adding a surfactant and fatty acid thereto; and
cooling the resulting mixture at room temperature.

47. The method according to claim 46, further comprising a step of milling after cooling at room temperature.
